(19) United States
(12) Patent Application Publication
(10) Pub. No.: US 2006/0234316 A1
(43) Pub. Date: Oct. 19, 2006

(54) Methods for distinguishing type-1 from type-2 diabetes

(76) Inventor: David H. Wagner, Denver, CO (US)

Correspondence Address: MORGAN LEWIS & BOCKIUS LLP, 1111 PENNSYLVANIA AVENUE NW, WASHINGTON, DC, 20004, US

(21) Appl. No.: 11/399,384

(22) Filed: Apr. 7, 2006

Related U.S. Application Data
(63) Continuation-in-part of application No. 10/563,570.
(60) Provisional application No. 60/484,655, filed on Jul. 7, 2003.

(51) Int. Cl. G01N 33/567

(52) U.S. Cl. 435/7.21

(57) ABSTRACT

The present invention provides a new method for the prediction of, or diagnosis of, auto-immune diseases, thereby alerting the subject to the presence of, or propensity to develop, an auto-immune disease so that preventative or therapeutic regiments may be initiated or changed so as to treat, modulate or prevent expansion of the CD4^{lo}CD40^{hi} T cell population responsible for the destructive inflammation. The invention also discloses agents which modulate, treat or prevent expansion of CD4^{lo}CD40^{hi} T cells. In one embodiment, the method is predictive of type 1 diabetes. Furthermore, the present invention provides a new method for distinguishing type 1 from type 2 diabetes.

### CROSS-REFERENCE TO RELATED APPLICATIONS

This application is a Continuation-in-Part of U.S. application Ser. No. 10/563,570 filed Jan. 6, 2006, which is a national phase application of PCT/US04/21646, filed Jul. 7, 2004, which claims the benefit of U.S. Provisional Application 60/484,655, filed Jul. 7, 2003, which are hereby incorporated by reference in their entirety.

### FIELD OF THE INVENTION

The invention relates to the fields of diagnosis and treatment of auto-immune diseases. More particularly, the present invention provides methods for determining the propensity to develop auto-immune disease(s), diagnosis of existing autoimmune diseases and provides methods and compositions for treatment of the auto-immune disease(s). The invention also provides methods for distinguishing type-1 from type-2 diabetes.

### BACKGROUND OF THE INVENTION

### Auto-Immune Diseases

Auto-immune diseases, regardless of the nature of the particular disease, arise because the immune system of an afflicted individual responds, inappropriately, to self-tissue, as though it were an infection. This response results in persistent and cumulatively destructive inflammation leading to irreversible tissue damage.

The auto-immune nature of the disease is that T cells of the immune system mediate the process. Furthermore, a unique classification of T cell characterized as auto-aggressive is responsible for the tissue damage. The population of T cells capable of becoming auto-aggressive has recently been identified (Wagner, D. H., Jr. et al., Int. J. Mol. Med. 4, 231-242 (1999); Wagner, D. H., Jr. et al., Proc. Natl. Acad. Sci. USA 99, 3782-7 (2002), and Vaitaitis, G. M. et al., Cutting Edge, J. Immunol. 170, 3455-459 (2003)). T cells can be identified by the expression of certain molecules including CD4 or CD8 and the T cell receptor, TCR. It has been determined that T cells which can be identified as auto-aggressive express the molecule CD40 (Wagner, D. H., Jr. et al., (1999); Wagner, D. H., Jr. et al., (2002), and Vaitaitis, G. M. et al., (2003)).

During a normal immune response, invading pathogens such as bacteria, fungi, parasites, viri or even neoplastic tissue including tumors are processed by specific cells of the immune system (macrophages, dendritic cells) and presented to T cells to initiate a response. These “foreign” pathogens are so identified because they are not part of the normal tissue of the individual. The T cell, through a protein on its cell surface, the T cell receptor (TCR), responds to the specific antigen being presented. There is a wide range of T cells, each expressing a specific receptor. In theory, one T cell has only one specific T cell receptor. Therefore, a T cell expressing its predetermined TCR encounters antigens that are being presented. The specialized antigen presenting cells (APC) of the immune system present antigens in the context of a cell surface protein, major-histocompatibility complex (MHC) class II, also known as Human Leukocyte Antigens (HLA). When a T cell recognizes the presented antigen, it becomes activated. The process of T cell activation includes induction of proliferation and production/secretion of proteins called cytokines that are able to assist the immune response. The cytokines recruit other lymphocytes to the infection, and help to activate cells involved in the destruction of the pathogen to establish localized inflammation and to ultimately resolve the infection.

Inflammation during infection is necessary and important to the removal of pathogens. It is only during auto-immune disease that persistent inflammation is damaging. It is necessary for an individual to maintain a collection of different TCR-expressing T cells, referred to as the T cell repertoire. This provides the necessary wide range of immunity.

While a variety of T cells provide an individual with normal immunity, in certain instances T cells arise which do not respond to foreign tissue but instead respond to an individual's self-tissue, resulting in an auto-immune disease. For instance in type 1 diabetes, afflicted patients generate T cells that react to the β-cells of the pancreatic islets. These T cells respond to antigens of the β-cells as though the cells were foreign, establishing inflammation and tissue destruction. In this case, the β cell ceases to produce insulin, a hormone necessary for normal metabolic functions, and clinical hyperglycemia (elevated glucose levels) ensues. In other auto-immune diseases, similar events occur, that is, T cells respond to self-tissue as though it was foreign. This interaction establishes inflammation and eventual tissue destruction.

### RAG Proteins in Auto-Immune Diseases

The process that generates TCR molecules involves a class of proteins termed recombination-activating-gene (RAG1 (SEQ ID NO: 2)) and RAG2 (SEQ ID NO. 4)) proteins. As T cells develop normally, the RAG proteins become activated to alter the genes for the TCR. This process occurs many times in the thymus, thus generating a wide variety of T cells capable of responding to antigens later in the periphery (Akamatsu, Y. & Oettinger, M. A., Mol. Cell. Biol. 18, 4670-8 (1998); Noordzij, J. et al., Blood 96, 203-209 (2000); and, Yannoutsos, N. et al., J. Exp. Med. 194, 471-80 (2001)).

The TCR is composed of α chain and β chain proteins (Malissen, M. et al., Immunology Today 13, 315-322 (1992); Chien, Y. H. & Davis, M. M., Immunology Today 14, 597-602 (1993)). Early during development of T cells within the thymus, the RAG proteins become activated, and migrate to the nucleus of the cell, where the proteins bind to DNA within the genes of the TCR β-chain, cut the DNA, and splice it back together in a way that alters the gene (Yannoutsos, N. et al., J. Exp. Med. 194, 471-80 (2001)). This is repeated for the α-chain gene. The process is repeated numerous times in developing T cells, and thus generates different TCR molecules, referred to as the T cell repertoire. The newly generated T cells then go through processes of positive and negative selection to remove any potentially damaging T cells (Nossal, G. J. V., Cell 76, 229-239 (1994); von Boehmer, H., Cell 76, 219-228 (1994)) including auto-aggressive T cells. The “safe” T cells then migrate to peripheral organs such as spleen, lymph nodes, lung, intestine, liver, etc. to await activation once a pathogen invades the body.

It has recently been shown (see, for example, U.S. Pat. No. 6,187,584) that RAG proteins contain D35E like motifs which are similar to the D35E motifs of retroviral integrases. U.S. Pat. No. 6,187,584 discloses a site-specific DNA binding site which is highly conserved and shared between the Herpes major DNA binding proteins, the RAG proteins, and the integrases of retroviruses. The highly conserved D35E motif may be subject to pharmacological modulation and agents interacting with the D35E motif may exhibit activity against retroviral integrases such as human immunodeficiency virus (HIV), and Herpes viruses, as well as immunomodulatory properties via interaction with RAG.

A recent report describes a new class of drugs, chaetochromins, capable of inhibiting the RAG proteins but in a non-cellular system (Melek, M. et al., Proc. Natl. Acad. Sci. USA 99, 134-7 (2002)). This class of drugs, also called “HIV Integrase Inhibitors,” have also been described elsewhere. See, for example, U.S. Pat. Nos. 6,403,347; 6,110,716; and, WO99/40183. These drugs have been shown to be inhibitors of human immunodeficiency virus (HIV) integration (Singh, S. B. et al., Org. Lett. 4, 1123-6 (2002); Singh, S. B. et al., J. Nat. Prod. 64, 874-82 (2001)) and are believed to act by inhibiting strand transfer and cleavage activity.

### Type-1 and Type-2 Diabetes

There are 20.8 million people in the United States, or 7% of the population, who have diabetes. While an estimated 14.6 million have been diagnosed with diabetes, 6.2 million people (or nearly one-third) are unaware that they have the disease. Diabetes is a disease characterized by the inability to produce or properly use insulin. Insulin is a hormone that is needed to convert sugar, starches and other food into energy needed for daily life. The cause of diabetes continues to be a mystery, although both genetics and environmental factors such as obesity and lack of exercise appear to play roles.

There are two major types of diabetes, called type 1 and type 2. Of all the patients with diabetes, only approximately 10% of the patients have type 1 diabetes and the remaining 90% have type 2 diabetes. Type 1 diabetes is also called insulin dependent diabetes mellitus (IDDM), or juvenile onset diabetes mellitus. In type 1 diabetes, the pancreas undergoes an autoimmune attack by the body itself, and is rendered incapable of making insulin. The patient with type 1 diabetes must continuously rely on insulin medication for survival.

In autoimmune diseases, such as type 1 diabetes, the immune system mistakenly manufactures antibodies and inflammatory cells that are directed against and cause damage to patients' own body tissues. It is believed that the tendency to develop these abnormal antibodies in type 1 diabetes is, in part, genetically inherited, though the details are not fully understood. Exposure to certain viral infections (mumps and Coxsackie viruses) or other environmental toxins may serve to trigger abnormal antibody responses that cause damage to the pancreas cells where insulin is made. These antibodies can be measured in the majority of patients, and may help determine which individuals are at risk for developing type 1 diabetes.

Unlike type 1 diabetes, type 2 diabetes is known as non-insulin dependent diabetes mellitus (NIDDM), or adult onset diabetes mellitus (AODM). In type 2 diabetes, patients can still produce insulin, but do so relatively inadequately for their body's needs, particularly in the face of insulin resistance as discussed above. In many cases this actually means the pancreas produces larger than normal quantities of insulin. A major feature of type 2 diabetes is a lack of sensitivity to insulin by the cells of the body (particularly fat and muscle cells). Thus, larger quantities of insulin are produced as an attempt to get these cells to recognize that insulin is, in fact, present. In addition to the problems with an increase in insulin resistance, the release of insulin by the pancreas may also be defective and suboptimal. In fact, there is a known steady decline in beta cell production of insulin in type 2 diabetes that contributes to worsening glucose control. This is a major factor for many patients with type 2 diabetes who ultimately require insulin therapy. Finally, the liver in these patients continues to produce glucose through a process called gluconeogenesis despite elevated glucose levels.

While it is said that type 2 diabetes occurs mostly in individuals over 30 years old and the incidence increases with age, the number patients with type 2 diabetes who are barely in their teen years has dramatically grown in recent years. In fact, for the first time in the history of humans, type 2 diabetes is now more common than type 1 diabetes in childhood. Most of these cases are a direct result of poor eating habits, higher body weight, and lack of exercise. While there is a strong genetic component to developing this form of diabetes, there are other risk factors, the most significant of which Is obesity. There is a direct relationship between the degree of obesity and the risk of developing type 2 diabetes, and this holds true in children as well as adults. It is estimated that the chance to develop diabetes doubles for every 20% increase over desirable body weight.

Regarding age, data shows that for each decade after 40 years of age regardless of weight there is an increase in incidence of diabetes. The prevalence of diabetes in persons 65 to 74 years of age is nearly 20%. Type 2 diabetes is more common in certain ethnic groups. Compared with a 6% prevalence in Caucasians, the prevalence in African Americans and Asian Americans is estimated to be 10%, in Hispanics 15%, and in certain Native American tribes 20% to 50%. Finally, diabetes occurs much more frequently in women with a prior history of diabetes that develops during pregnancy. Type 2 diabetes is often associated with a strong familial, probably genetic predisposition. This is less common in the autoimmune form of type 1 diabetes.

### Role of CD4 and CD40 in Auto-Immune Diseases

The importance of CD40 in auto-immune diseases, including collagen-induced arthritis (Durie, F. H. et al., Science 281, 1328-1330 (1993)), chronic inflammatory diseases, including colitis (De Jong, Y. et al., Gastroenterology 119, 715-723 (2000)), atherosclerosis (Lutgens, E. et al., Nat. Med. 5, 1313-6 (1999)), and systemic lupus erythematosus (Wang, X. et al., J. Immunol. 168, 2046-53 (2002)) among others, continues to be expounded. It has been shown that blocking CD40-CD40 ligand (CD154) (SEQ ID NO: 6) interaction prevents rejection of islet transplants (Zheng, X. X. et al., Transplant Proc 31, 627-8 (1999); Molano, R. D. et al., Transplant Proc 33, 248-9 (2001)). T cell infiltration into the pancreas occurs in NOD mice as early as 3-4 weeks of age with extensive insulitis at 12-weeks of age (Luhder, F. et al., J. Exp. Med. 187, 379-87 (1998)). Injecting 3-week old NOD mice with CD40 Ligand (CD154) blocking antibodies prevented onset of T1D but injecting NOD mice at 9-weeks of age had no effect on disease onset (Balasa, B. et al., J. Immunol. 159, 4620-7 (1997)). This suggests an important cellular developmental framework with regards to CD40 and diabetes that potentially involves T cells.

In a diabetes animal model system, CD4⁺ T cells which also express the CD40 molecule have been shown to be pathogenic. Isolation and purification of these cells repeatedly transfers diabetes to non-sick animals, whereas other CD4⁺ cells that do not express the CD40 molecule do not transfer disease (Wagner, D. H., Jr. et al., (2002)). Furthermore, the pathogenic T cells have been shown to express lower levels of the CD4 molecule. It was previously shown that numerous auto-immune prone animal strains have elevated numbers of CD40-expressing CD4 T cells (Wagner, D. H., Jr. et al., (1999)). Other studies have deomonstrated that humans have CD40-expressing T cells. Individuals that were heavy smokers or tobacco users and therefore more susceptible to respiratory disease had higher numbers of CD40-expressing T cells, consistent with the involvement of CD40-expressing T cells in disease. The mechanism by which these T cells generate TCR molecules that respond to self-tissue (Vaitaitis, G. M. et al., (2003)) has been determined. A subpopulation of T cells categorized by expression of CD40 has been discovered to be auto-aggressive. By engaging the CD40 molecule, the RAG proteins can be activated again. That is, activation of the RAG proteins occur in peripheral T cells after the initial activation of RAG proteins during T cell development. This process causes a new TCR molecule to be expressed on the surface of the T cell (Vaitaitis, G. M. et al., (2003)).

Numerous drugs are available to treat the symptoms of auto-immunity but as yet there is no approach to predict, modulate or prevent expansion of the cells responsible for the diseases and destructive inflammation. Also, a rapid test for distinguishing between type 1 and type 2 diabetes is not currently available. Thus, in view of the problems with the known drugs, treatment and diagnostic methods discussed above, new drugs and new methods for the prediction, diagnosis, modulation and treatment of auto-immune diseases are needed.

### SUMMARY OF THE INVENTION

The present invention solves the problems discussed above and provides a new type of drug to treat the symptoms of auto-immunity. The new type of drug disclosed herein modulates, treats or prevents expansion of the cells responsible for the auto-immune disease and the destructive inflammation they cause. The present invention also provides a new method for the prediction of, or diagnosis of, auto-immune diseases, thereby alerting the subject to the presence of, or propensity to develop, an auto-immune disease so that preventive or therapeutic regimens may be initiated or changed which will treat, modulate or prevent expansion of the cell population responsible for the destructive inflammation. Additionally, the present invention provides a method to differenciate type-1 from type-2 diabetes so that therapeutic regimens may be initiated or changed which will treat and/or modulate the disease.

The invention herein includes a method for determining whether a test subject has at least one auto-immune disease comprising a) obtaining blood from the predetermined test subject thus obtaining a test sample; b) obtaining blood from a non-autoimmune subject thus obtaining a control sample; c) contacting the test sample and the control sample with a combination of at least one detectably-labeled anti-CD4 antibody and a least one detectably-labeled anti-CD40 antibody; d) detecting the level of CD4^{lo}CD40^{hi} T cells in the test sample and in the control sample; wherein when there is an increase in the level of CD4^{lo}CD40^{hi} T cells in the test sample as compared to the level of CD4^{lo}CD40^{hi} T cells in the control sample, the test subject has at least one auto-immune disease.

The invention herein also includes a method for determining whether a predetermined test subject is susceptible to developing at least one predetermined auto-immune disease comprising a) obtaining a first sample of blood from said predetermined test subject; b) obtaining a second sample of blood from said same subject; c) detecting the CD4^{lo}CD40^{hi} T cell population in said first and second samples; d) contacting said second test sample with at least one predetermined antigen indicative of at least one predetermined auto-immune disease for a length of time and in an amount sufficient to obtain a positive or negative cellular response in the CD4^{lo}CD40^{hi} T cell population of said second sample, e) determining whether a positive or negative cellular response occurs in the CD4^{lo}CD40^{hi} T cell population of said first and said second samples by measuring at least one response selected from the group consisting of CD4^{lo}CD40^{hi} T cell proliferation, CD4^{lo}CD40^{hi} T cell death and CD4^{lo}CD40^{hi} cytokine production, wherein when a positive response occurs in the CD4^{lo}CD40^{hi} T cell population of the second sample as compared to the response in the CD4^{lo}CD40^{hi} T cell population from the first sample, the predetermined subject is susceptible to developing the at least one predetermined autoimmune disease.

The invention is also directed to a method of modulating the proliferation of CD4^{lo}CD40^{hi} T cells in a subject in need of said modulation comprising at least one method selected from the group consisting of a) contacting said subject with at least one agent which inhibits the activation of RAG recombinase activity; b) contacting said subject with an antibody molecule, or fragment thereof, to CD40; c) contacting said subject with an antibody molecule, or fragment thereof, to CD154; d) contacting said subject with at least one blocking peptide to prevent interaction of the CD40 receptor with the CD154 ligand; e) contacting said subject with at least one RNA molecule specifically hybridizing to the RAG2 gene product; and, f) contacting said subject with at least one RNA molecule specifically hybridizing to the RAG1 gene product; wherein said contacting is for a length of time sufficient and in an amount sufficient to modulate the proliferation of CD4^{lo}CD40^{hi} T cells in said subject.

The invention is also directed to a kit for detecting CD4^{lo}CD40^{hi} T cells comprising a) at least one detectably labeled anti-CD4 antibody and at least one detectably labeled anti-CD40 antibody; and, b) at least one predetermined antigen indicative of at least one predetermined auto-immune disease.

The invention herein also includes a method for differentiating whether a test subject has type-1 or type-2 diabetes by a) obtaining a blood sample from the test subject; b) detecting CD4⁺CD40⁺T cells in the blood sample; and c) determining whether the CD4⁺CD40⁺ T cells in the blood sample are expanded by comparing to CD4⁺CD40⁺ T cells in a control sample of blood, wherein an expanded level of CD4⁺CD40⁺ T cells indicate that the subject has type-1 diabetes and not type-2 diabetes.

The invention is also directed to a kit for determining whether a subject has type-1 or type-2 diabetes comprising: a) a control blood sample; b) means for obtaining a blood smaple from the subject; c) means for detecting CD4⁺CD40⁺ T cells; and d) a means for determining whether the CD4⁺CD40⁺ T cells in the blood sample from the subject are expanded.

### BRIEF DESCRIPTION OF THE DRAWINGS

The foregoing summary, as well as the following detailed description of the invention, will be better understood when read in conjunction with the appended figures. For the purpose of illustrating the invention, shown in the figures are embodiments which are presently preferred. It should be understood, however, that the invention is not limited to the precise arrangements, examples and instrumentalities shown.

FIGS. 1A-B. Auto-aggressive T cells expand as diabetes-prone mice age. (A) Expression of CD4⁺ and CD40⁺ on T cells of NOD mice at 3 weeks, 6 weeks, 12 weeks and 18 weeks. (B) Expression of CD4⁺ and CD40⁺ on T cells of NOD mice at 12 weeks after CD40-CD154 interaction is blocked.

FIGS. 2A-C. Highly purified CD40⁺ T cells transfer diabetes. (A). CD4⁺CD40⁺ T cells or CD4⁺CD40⁻ T cells from diabetic NOD (line with diamonds) or from pre-diabetic NOD mice (line with squares) rapidly transfers diabetes. Half of the CD40⁺ recipients became diabetic, blood glucose (b.g.)>250 mg/ml at 10 days post injection and the remaining were diabetic by 14 days. CD40⁻ T cell recipients did not develop diabetes through 45 days. Half of the animals receiving CD4⁺CD40⁺ T cells purified from pre-diabetic mice became diabetic at 12 days, and all animals were diabetic by 15 days, with none of the CD4⁺CD40⁻ recipients becoming diabetic (p<0.05). (B). Pancreata of NOD.scid animals receiving CD4⁺CD40⁺ T cells demonstrate T cell infiltration and overall lack of insulin granules while (C), pancreata of CD4⁺CD40⁻ T cell recipients show no T cell infiltration. Islet infiltration was scored with >100 islets/treatment-group examined. CD40⁺ recipients demonstrated extensive infiltration with >95% of islets infiltrated whereas CD40⁻ recipients had no detectable infiltrate at 15 days. Panels shown are representative of all experiments.

FIGS. 3A-C. Expansions in CD4^{lo}CD40⁺ T cells as NOD mice develop. CD4 versus CD40 T cell levels in T cells from (A) NOD, (B) NOR and (C) BALB/c mice at 3-weeks, 6-weeks, 12-weeks and 18-weeks of age (Data was verified from CD3 magnetic column, Miltenyi Corp., purified cells). Gates were set from isotype controls. (A) In NOD mice at 3-weeks the CD4^{lo}CD40⁺ population is 6% of total T cells, at 6-weeks, CD4^{lo}CD40⁺ are 15% of total T cells, at 12-weeks CD4^{lo}CD40⁺ are 25%, and at 18-weeks CD4^{lo}CD40⁺ are 40% of T cells. (B) In NOR mice at 6-weeks, CD4^{lo}CD40⁺ are 15% of total T cells, at 12-weeks CD4^{lo}CD40⁺ are 15%, and at 18-weeks CD4^{lo}CD40⁺ are 12% of T cells. NORs at 3-weeks were not available. (C) In BALB/c mice at 3-weeks the CD4^{lo}CD40⁺ population is 16% of total T cells, at 6-weeks, 8% of total T cells, at 12-weeks 6%, and at 18-weeks CD4^{lo}CD40⁺ are 5% of T cells. Data represent 3 separate experiments.

FIGS. 4A-C. CD40 driven expansions of specific Vα⁺ T cells in NOD mice. Vα⁺ T cells within the CD4^{lo}CD40⁺ T cell population were determined in immediately ex vivo T cells or CD40-crosslinked T cells from (A) NOD, (B) NOR and (C) BALB/c mice, at age 3-weeks, 12-weeks and 18-weeks. Untreated (light bars) or CD40 crosslinked for 18 hrs (dark bars) T cells are represented. Data are percent Vα⁺ T cells only within the CD4^{lo}CD40⁺ gated populations above appropriate isotype controls. Data are an average of 3 experiments with 3 animals in each experiment; x-axis is percent Vα⁺ in gated CD4^{lo}CD40⁺ T cells.

FIGS. 5A-D. Expansions of Vα3.2⁺ T cells in pancreata of pre-diabetic and diabetic NOD mice. Pancreata from (A) 12-week old pre-diabetic (n=4), and (B)>18-week old diabetic NOD (n=4) show expansions of Vα3.2⁺ and Vα8.3⁺ T cells within the gated CD4⁺CD40⁺ population, above isotype controls. (C) T cells from CD4⁺CD40⁺ NOD.scid recipients and from CD4⁺CD40⁻ NOD.scid recipient at 15 days post injections demonstrate Vα⁺ expansion (solid lines) above isotype controls (dashed lines). (D) T cells from CD4⁺CD40⁻ NOD.scid recipients at 15 days post injections demonstrate no significant Vα⁺ expansions. As in FIG. 2, data represent 3 separate experiments, n=12 for each treatment. FIG. 5 demonstrates that during autoimmune diabetes, type-1, there are expansion of specific Vα⁺ T cells. The numbering system is arbitrary. We have identified CD40⁺ T cells in humans and predict there will be specific Vα⁺ expansions.

FIGS. 6A-C. Pancreatic histology from Vα3.2⁺ and Vα8.3⁺ NOD.scid recipients. (A) Vα3.2⁺ T cells transfer diabetes but Vα8.3⁺ T cells do not. Vα3.2⁺ T cells were >80% CD40⁺ while only 30% of Vα8.3⁺ T cells were CD40⁺. As controls, CD40-depleted T cells did not transfer diabetes. As before, diabetes was considered to be a blood glucose >150 mg/ml. Pancreata from (B) Vα3.2⁺ recipients demonstrate extensive infiltration and lack of insulin production. (C) Vα8.3⁺ recipients did not demonstrate infiltrated islets.

FIGS. 7A-B. CD4⁺CD40⁺ T cell increases are predictive of rheumatoid arthritis. 7A. Rheumatoid arthritis patient. 7B. Control patient. See Example 4 for details.

FIGS. 8A-B. CD4⁺CD40⁺ T cell increases are predictive of asthma. 8A. Control patient. 8B. Asthma patient. See Example 5 for details.

FIGS. 9A-C. CD4⁺CD40⁺ T cells are predictive for human type I diabetes. FIG. 9A. Non-Diabetic human patient. FIG. 9B. Diabetic human patient. FIG. 9C. % CD4⁺CD40⁺ T cells in diabetic versus non-diabetic patients. See Example 6 for details.

FIGS. 10A-F. CD4^{lo}CD40⁺ T cells in Human T1D. Peripheral blood was analyzed in a blinded study. Blood samples were obtained from the Barbara Davis Center and stained for CD40/CD3/CD4 or CD40/CD8/CD4 expression. After analysis samples were unblinded. CD4^{lo}CD40⁺ and CD4^{hi}CD40⁻ T cell levels were demonstrated in T1D (A) and non-autoimmune control patients (B). CD4^{lo}CD40⁺ T cells were confirmed by counter staining with CD3, then gating on CD3⁺ T cells in T1D (E) and normal patients (F). T cells were stained for CD8 versus CD40. CD8⁺CD40⁺ T cells were determined by gating on CD3 as well (C and D). Dot plots are representative of a total of 25 T1D and 25 control patients.

FIGS. 11A-C. T1D CD4⁺CD40⁺ T Cells Are Diabetes-Related Self-Antigen Responsive. Histograms represent response of gated CD4^{lo}CD40⁺ (A) or CD4^{hi}CD40⁻ (B) T cells from T1D and control patients. T cells were exposed to APC alone (background) or APC+tetanus toxin for 48 hr. Numbers are percent of cells proliferating, measured as a loss of CFSE mean fluorescent intensity (MFI). Response of CD4^{lo}CD40⁺ or CD4^{hi}CD40⁻ (C) T cells from T1D and control patients to presented self-antigens is shown. Values reported are percent of CD4^{lo}CD40⁺ and CD4^{hi}CD40⁻ T cells with a decreased MFI in FL-1 after 96 hr of exposure. Cells were not anergic as determined by response to PHA. Values were graphed to demonstrate overall response to each antigen. Data are representative of 15 T1D and 12 control patients. Experiment was repeated 3 times. Response of CD4⁺CD40⁺ T cells from T1D to each self-antigen tested was significantly different from background (p<0.001 by T-test). Response of CD4⁺CD40⁻ T cells from both T1D and control patients, was not significantly different from background (p>0.25). PPI: pre-pro insulin; GAD: glutamic acid decarboxylase, TT: tetanus toxoid.

FIGS. 12A-B. Expanded CD4⁺CD40⁺ T cells in T1D Correlate to Diabetes Associated HLA-DR phenotypes and Is Independent of Hyperglycemia. (A) CD4⁺CD40⁺ T cell levels were determined as described in FIG. 1. In blinded studies, levels of CD4⁺CD40⁺ T cells were determined then the HLA-DR phenotype was revealed. Analysis determined the percentage of CD4⁺CD40⁺ T cells relative to each DR with both alleles reported for each patient. DR subsets were grouped as DR3/DR3; DR4/DR4; DR3/DR4; DR4/x with x corresponding to any DR other than DR4 or DR3; DR3/x with x corresponding to any DR other than DR4 or DR3; DRx/DRx with x corresponding to any DR other than DR4 or DR3. Diabetic patients are represented with diamonds and control patients with circles. The shaded area is the standard deviation range of CD4⁺CD40⁺ T cell percentages in non-autoimmune, non-diabetic patients. Statistics were done using the Student's T test comparison between means analysis. (B) The age range of patients was 19-64 years for T1D and 20-55 for controls. Percentages of CD4⁺CD40⁺ T cells from T1D versus controls were grouped by decades as 19-29; 30-39; and greater than 40. There were fewer patients aged 50 and over. There was no significant difference within the T1D age groups.

FIGS. 13A-B. CD4⁺CD40⁺ T Cells Remain Static in Type 2 Diabetes. (A) PBL were isolated from T1D, T2D and control patients as described in FIG. 1. Cells were isolated from ficoll-hypaque gradients, washed with PBS then stained with directly conjugated anti-CD40 (FITC) and anti-CD4 (CyChrome). For analysis quadrant gates were determined from isotype controls. Dot plots are representative of three separate experiments. (B) The peripheral blood percentage of CD4⁺CD40⁺ T cells from clinically diagnosed T1D, T2D or normal controls were determined and plotted. Mean levels were 17.1% in T2D patients (n=15); 18.8% in control (n=25) and 44.2% in T1D patients (n=25).

### DETAILED DESCRIPTION

General Description

The invention herein includes a method for determining whether a test subject has at least one auto-immune disease comprising a) obtaining blood from the predetermined test subject thus obtaining a test sample; b) obtaining blood from a non-autoimmune subject thus obtaining a control sample; c) contacting the test sample and the control sample with a combination of at least one detectably-labeled anti-CD4 antibody and at least one detectably-labeled anti-CD40 antibody; d) detecting the level of CD4^{lo}CD40^{hi} T cells in the test sample and in the control sample; wherein when there is an increase in the level of CD4^{lo}CD40^{hi} T cells in the test sample as compared to the level of CD4^{lo}CD40^{hi} T cells in the control sample, the test subject has at least one auto-immune disease. In one embodiment, the method further comprises isolating the test sample CD4^{lo}CD40^{hi} T cells and the control sample CD4^{lo}CD40^{hi} T cells from part 1d) and determining the presence or absence of an increase in production of at least one cytokine in the test T cell population as compared to the sample T cell population. In another embodiment of the method, the cytokine is at least one cytokine selected from the group consisting of Il-2, IL-4, IL-6, IL-10, TGFβ and IFNγ. In a different embodiment of the method, the auto-immune disease is selected from the group consisting of type 1 diabetes, rheumatoid arthritis, lupus, multiple sclerosis, atherosclerosis, Crohn's colitis, ulcerative gastritis, primary biliary cirrhosis, chronic obstructive pulmonary disease (COPD) and scleroderma. In a preferred embodiment, the auto-immune disease is type 1 diabetes. In a highly preferred embodiment, the COPD disease is emphysema. In one aspect of the invention, the detecting is by flowcytometry. In a highly preferred embodiment of the method, the subject is human.

The invention here in also includes a method for determining whether a predetermined test subject is susceptible to developing at least one predetermined auto-immune disease comprising a) obtaining a first sample of blood from said predetermined test subject; b) obtaining a second sample of blood from said same subject; c) detecting the CD4^{lo}CD40^{hi} T cell population in said first and second samples; d) contacting said second test sample with at least one predetermined antigen indicative of at least one predetermined auto-immune disease for a length of time and in an amount sufficient to obtain a positive or negative cellular response in the CD4^{lo}CD40^{hi} T cell population of said second sample, e) determining whether a positive or negative cellular response occurs in the CD4^{lo}CD40^{hi} T cell population of said first and said second samples by measuring at least one response selected from the group consisting of CD4^{lo}CD40^{hi} T cell proliferation, CD4^{lo}CD40^{hi} T cell death and CD4^{lo}CD40^{hi} cytokine production, wherein when a positive response occurs in the CD4^{lo}CD40^{hi} T cell population of the second sample as compared to the response from the CD4^{lo}CD40^{hi} T cell population of the first sample, the predetermined subject is susceptible to developing the at least one predetermined autoimmune disease. In one embodiment, the T cells are isolated or purified from the first sample, the second sample or both samples. In one embodiment of the method, a positive response is an increase in CD4^{lo}CD40^{hi} T cell proliferation, an increase in CD4^{lo}CD40^{hi} T cell death and an increase in production of at least one cytokine produced by said CD4^{lo}CD40^{hi} T cell population. In a different embodiment of the method, the at least one cytokine is selected from the group consisting of IL-2, IL-4, IL-6, IL-10, TGFβ and IFNγ. In a preferred embodiment of the method, the at least one preselected auto-immune disease is type 1 diabetes and said antigen is pancreatic tissue. In another embodiment, the at least one preselected auto-immune disease is rheumatoid arthritis and said antigen is synovial tissue. In different embodiment of the method, the at least one preselected auto-immune disease is multiple sclerosis and said antigen is nervous tissue. In yet another embodiment of the method, the at least one preselected auto-immune disease is scleroderma and said antigen is skin tissue. In an additional embodiment, the at least one auto-immune disease is atherosclerosis and said antigen is cardiac tissue. In a highly preferred embodiment of the method, the subject is human.

The invention is also directed to a method of modulating the proliferation of CD4^{lo}CD40^{hi} T cells in a subject in need of said modulation comprising at least one method selected from the group consisting of a) contacting said subject with at least one agent which inhibits the activation of RAG recombinase activity; b) contacting said subject with an antibody molecule, or fragment thereof, to CD40; c) contacting said subject with an antibody molecule, or fragment thereof, to CD154; d) contacting said subject with at least one blocking peptide to prevent interaction of the CD40 receptor with the CD154 ligand; e) contacting said subject with at least one RNA molecule specifically hybridizing to the RAG2 gene product; and, f) contacting said subject with at least one RNA molecule specifically hybridizing to the RAG1 gene product; wherein said contacting is for a length of time sufficient and in an amount sufficient to modulate the proliferation of CD4^{lo}CD40^{hi} T cells in said subject. In one embodiment of the method of in part a), at least one agent is a chaetochromin or a derivative thereof. In another embodiment of the method, in part b), the antibody fragment is an Fab portion. In a different embodiment of the method, in part c), the antibody fragment is an Fab portion. In yet a different embodiment, in part d), the blocking peptide is selected from the group consisting of SSKTTSVLQWAEKGYYTMSNNLVT (SEQ ID NO: 7) and QIAAHVISEASSK (SEQ ID NO: 8). In another embodiment, in part e), the RNA molecule is selected from the group consisting of

| | |
|---|---|
| 5′-AUGUCUCUGCAGAUGGUAACdAdG-3′; | (SEQ ID NO:9) |
| 5′-CUGUUACCAUCUGCAGAGACdAdU-3′; | (SEQ ID NO:10) |
| 5′GGUAGGAGAUCUUCCUG AAGdCdC-3′; | (SEQ ID NO:11) |
| 5′GGGGAUGGGCACUGGGUCCAUGdCdU-3′; | (SEQ ID NO:12) |
| 5′AGCAUGGACCCAGUGCCCAUGCdCdC-3′; | (SEQ ID NO:13) |
| and, | |
| 5′-CUGUUACCAUCUGCA GAGACdAdU-3′. | (SEQ ID NO:14) |

In yet another embodiment of the method, in part f), the RNA molecule is selected from the group consisting of

| | |
|---|---|
| 5′-AUGGCAGCCUCUUUCCCACCCAdCdC-3′; | (SEQ ID NO:15) |
| 5′-GGUGGGUGGGAAAGAGGCUGCCdAdU-3′; | (SEQ ID NO:16) |
| 5′-AAACUUGCAGCUCAGCAAAAAACdTdC-3′; | (SEQ ID NO:17) |
| 5′-GAGUUUUUUGCUGAGCUGCAAGUUdUdU-3′; | (SEQ ID NO:18) |
| 5′-GAGUUUUUUGCUGAGCUGCAAGUUdUdU-3′; | (SEQ ID NO:19) |
| 5′-UCACAAAACCCUGGCCCAUGUUdCdC-3′; | (SEQ ID NO:20) |
| and, | |
| 5′-GGAACAUGGGCCAGGGUUUUGUdGdA-3′. | (SEQ ID NO:21) |

In a different embodiment of the method, the subject has an increased level of CD4^{lo}CD40^{hi} T cells as compared to the level of CD4^{lo}CD40^{hi} T cells in a non-auto-immune subject and the modulation is a decrease in the level of CD4^{lo}CD40^{hi} Tcells. In a highly preferred embodiment of the method, the subject is human.

The invention is also directed to a kit for detecting CD4^{lo}CD40^{hi} T cells comprising a) at least one detectably labeled anti-CD4 antibody and at least one detectably labeled anti-CD40 antibody; and, b) at least one predetermined antigen indicative of at least one predetermined auto-immune disease.

The present invention is based in part on the finding that in the NOD mouse type 1 diabetes (T1D) model, CD4⁺CD40⁺ T cells comprise a unique pathogenic T cell subset that expand concurrently with progressive insulitis and demonstrate pathogenesis by transferring T1D to NOD.scid recipients. Recent studies demonstrate that pancreatic lymph nodes of human T1D patients contain diabetes-auto-antigen responsive T cells with a limited TCR repertoire. The present invention describes a unique T cell subset that is drastically expanded in human T1D patients, including recent onset and long-term diagnosed patients compared to non-autoimmune controls. In T1D but not in control patients, CD4⁺CD40⁺ T cells are responsive to human islets, pre-pro-insulin and GAD peptides. Expanded CD4⁺CD40⁺ T cells correlate with T1D associated HLA-DR4 and DR3 alleles, and expanded levels occur independently of age.

Although any methods and materials similar or equivalent to those described herein can be used in the practice or testing of the present invention, the preferred methods and materials are described.

Definitions

Unless defined otherwise, all technical and scientific terms used herein have the same meaning as commonly understood by one of ordinary skill in the art to which this invention belongs.

As used herein, the term “agent” refers to any compound which is pharmacologically and/or biologically active in a subject.

As used herein, the term “antibody” refers to intact immunoglobulins. “Antibody fragments” refers to a number of well characterized fragments produced by digestion with various peptidases. Thus, for example, pepsin digests an antibody below the disulfide linkages in the hinge region to produce F(ab)′₂ a dimer of Fab which itself is a light chain joined to V_{H} C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Fundamental Immunology, Third Edition, W. E. Paul, ed., Raven Press, N.Y. 1993). While various antibody fragments are defined in terms of the digestion of an intact antibody, such fragments may be synthesized de novo either chemically or by utilizing recombinant DNA methodology. Thus, the term “antibody fragments” includes antibody fragments either produced by the modification of whole antibodies or those synthesized de novo using recombinant DNA methodologies, such as, for example, single chain Fv. See, for example, U.S. Pat. No. 6,552,181.

As used herein, the term “amplifier” is used in reference to a system which enhances the signal in a detection method, such as an ELISA (e.g., an alkaline phosphatase amplifier system used in an ELISA).

As used herein, the term “auto-aggressive T cells” refers to a population of T cells which stain positively for both the CD4⁺ and CD40⁺ markers. These cells exist in some low level in normal individuals but are increased in numbers in individuals expressing, or prone to developing, auto-immune diseases.

As used herein, the term “auto-immune” disease refers to a disease or condition where the target of the disease is “self” or a “self antigen.” There are a number of diseases that are believed to involve T cell immunity directed to self antigens. The auto-immune disease may be triggered directly or indirectly by one or more antigens.

As used herein, the term “CD4⁺” refers to a cell surface molecule the presence or absence of which is used to describe and characterize a specific population of T cells. For example, a cell population expressing low levels of CD4 is termed “CD4^{+lo}”, a cell population expressing hi levels of CD4 is termed “CD4^{+hi}”, and a cell population which is not detectably expressing, for example, CD4, is termed “CD4⁻”.

As used herein, the term “CD40⁺ cell” refers to a cell surface molecule the presence or absence of which is used to describe and characterize a specific population of T cells. For example, a cell population expressing low levels of CD40 is termed “CD40^{+lo}”; a cell population expressing high levels of CD4 is termed “CD40^{+hi}”, and a cell population which is not detectably expressing CD40 is termed “CD40⁻”.

As used herein, the term “CD4⁺CD40⁺” refers to the T cells expressing low levels of CD4 and high levels of CD40. The term “CD4⁺CD40⁺” refers to the same cell population as the term “CD4^{lo}CD40⁺.”

As used herein, the term “CD154” refers to a cell surface molecule which is a ligand for the CD40 receptor.

As used herein, the term “contacting with at least one agent” should be understood to mean providing an agent of the invention or a prodrug of an agent of the invention to a subject.

As used herein, the term “capture antibody” refers to an antibody that is used in a sandwich ELISA to bind (i.e., capture) an antigen in a sample prior to detection of the antigen. For example, in some embodiments, the polyclonal anti-βA-MDA antibody of the present invention serves as a capture antibody when immobilized in a microtiter plate well. This capture antibody binds βA-MDA antigen present in a sample added to the well. In one embodiment of the present invention, biotinylated capture antibodies are used in the present invention in conjunction with avidin-coated solid support. Another antibody (i.e., the detection antibody) is then used to bind and detect the antigen-antibody complex, in effect forming a “sandwich” comprised of antibody-antigen-antibody (i.e., a sandwich ELISA).

As used herein, a “detection antibody” is an antibody which carries a means for visualization or quantitation, which is typically a conjugated enzyme moiety that typically yields a colored or fluorescent reaction product following the addition of a suitable substrate. Conjugated enzymes commonly used with detection antibodies in the ELISA include horseradish peroxidase, urease, alkaline phosphatase, glucoamylase and β-galactosidase. In some embodiments, the detection antibody is directed against the antigen of interest, while in other embodiments, the detection antibody is not directed against the antigen of interest. In some embodiments, the detection antibody is an anti-species antibody. Alternatively, the detection antibody is prepared with a label such as biotin, a fluorescent marker, or a radioisotope, and is detected and/or quantitated using this label.

As used herein, the term “derivative thereof” refers to a chemically modified agent wherein the chemical modification takes place at one or more functional groups of the agent and/or on an aromatic ring, when present. The derivative however is expected to retain the pharmacological activity of the agent from which it is derived.

As used herein, the term “detecting” refers to assaying, measuring, discovering or discerning the existence, presence or fact of a predetermined target entity, for example, CD4 or CD40.

As used herein, the term “detectably labeled” refers to any substance whose detection or measurement, either directly or indirectly, by physical or chemical means, is indicative of the presence of the target entity, for example, CD4 and CD40 in the test sample. Many detectable labels are known in the art and useful in the practice of the invention.

As used herein, the term “disease specific antigen” refers to one or more antigens known to be related to, involved with, or expressed during the existence of, a specific auto-immune disease. For example, human insulinoma cells or pancreatic tissue obtained from a pancreatic biopsy express one or more antigens specific for type 1 diabetes. Another example of an antigen which is specific for an autoimmune disease is myelin basic protein, specific for multiple sclerosis. There are numerous citations in the literature of T cells responding to whole tissue which is sufficiently descriptive for autoimmunity. See, for example, Haskins, G. E. & and Records, R. E., Nebr. Med. J. 67, 23 (1982); Haskins, K. M., et al., Proc. Natl. Acad. Sci. USA 86, 8000 (1989); Haskins, K. & McDuffie, M., Science 249, 1433 (1990); and Haskins, K. & Wegmann, D., Diabetes 45, 1299 (1996).

As used herein, the term “ELISA” refers to enzyme-linked immunosorbent assay (or EIA). Numerous ELISA methods and applications are known in the art, and are described in many references (See, e.g., Crowther, “Enzyme-Linked Immunosorbent Assay (ELISA),” in Molecular Biomethods Handbook, Rapley et al. [eds.], pp. 595-617, Humana Press, Inc., Totowa, N.J. [1998]; Harlow and Lane (eds.), Antibodies: A Laboratory Manual, Cold Spring Harbor Laboratory Press [1988]; Ausubel et al. (eds.), Current Protocols in Molecular Biology, Ch. 11, John Wiley & Sons, Inc., New York [1994]). In addition, there are numerous commercially available ELISA test systems.

As used herein, the term “immunoassay” refers to any assay that uses at least one specific antibody for the detection or quantitation of an antigen. Immunoassays include, but are not limited to, Western blots, ELISAs, radio-immunoassays, and immunofluorescence assays. Furthermore, many different ELISA formats are known to those in the art, any of which will find use in the present invention. However, it is not intended that the present invention be limited to these assays. In additional embodiments, other antigen-antibody reactions are used in the present invention, including but not limited to “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen), “complement fixation” (i.e., the use of complement in an antibody-antigen reaction method), and other methods commonly used in serology, immunology, immunocytochemistry, immunohistochemistry, and related fields.

As used herein, the term “propensity to develop” refers to the susceptibility, predisposition or liklihood that a particular subject will develop an auto-immune disease. Subjects susceptible to developing an auto-immune disease are also termed “auto-immune prone.” Such subjects do not exhibit detectable symptoms of an existing auto-immune disease. The auto-immune disease may not have yet developed, is inactive, or has not progressed to the point where symptoms or indications are exhibited by the subject, in which case the test is predictive of developing or expressing the auto-immune disease.

As used herein, the term “peripheral blood” means blood found in the vasculature.

As used herein, the terms “RAG1” or “RAG2” refer to proteins which interact with the recombination-activation-genes (“RAG”). (Li, T. T. et al., Eur. J. Immunol. 32 (10), 2792-2799 (2002); Schatz, D. G. et al., Cell 59 (6), 1035-1048 (1989)).

As used herein, the term “recombinogenic” refers to the ability to catalyze or otherwise be involved with or effect recombination of nucleic acid molecules. Specifically, such recombination could include, but is not limited to DNA strand breakage and DNA strand transfer, and transposition of mobile elements. See, for example, U.S. Pat. No. 6,187,584.

As used herein, the terms “reporter reagent,” “reporter molecule,” “detection substrate” and “detection reagent” are used in reference to reagents which permit the detection and/or quantitation of an antibody bound to an antigen. For example, in some embodiments, the reporter reagent is a calorimetric substrate for an enzyme that has been conjugated to an antibody. Addition of a suitable substrate to the antibody-enzyme conjugate results in the production of a calorimetric or fluorimetric signal (e.g., following the binding of the conjugated antibody to the antigen of interest). Other reporter reagents include, but are not limited to, radioactive compounds. This definition also encompasses the use of biotin and avidin-based compounds (e.g., including but not limited to neutravidin and streptavidin) as part of the detection system.

As used herein, the term “signal” is used generally in reference to any detectable process that indicates that a reaction has occurred, for example, binding of antibody to antigen. It is contemplated that signals in the form of radioactivity, fluorimetric or calorimetric products/reagents will all find use with the present invention. In various embodiments of the present invention, the signal is assessed qualitatively, while in alternative embodiments, the signal is assessed quantitatively.

As used herein, the term “solid support” is used in reference to any solid or stationary material to which reagents such as antibodies, antigens, and other test components are attached. For example, in the ELISA method, the wells of microtiter plates provide solid supports. Other examples of solid supports include microscope slides, coverslips, beads, particles, cell culture flasks, as well as many other suitable items.

As used herein, the term “subject” refers to an individual or patient. The subject can be any animal having or not having, predisposed or not predisposed, to developing, an auto-immune disease. Preferred subjects include humans and mammals.

I. Tests for Auto-Immune Diseases

A. Diagnostic Tests

1. Predetermined Auto-Immune Diseases

This invention specifically includes blood tests utilizing the characterization of auto-aggressive T cells by expression of both CD40 and low-level expression of CD4, thereby defining a new cell type. Diagnostic tests for known auto-immune diseases may be established according to the methods disclosed in this invention. The auto-immune disease may be active in a subject, in which case the test is diagnostic. This invention will diagnose known existing auto-immune diseases such as type 1 diabetes, rheumatoid arthritis, lupus, atherosclerosis, multiple sclerosis, Crohn's colitis, ulcerative gastritis, primary biliary cirrhosis and auto-immune hepatitis, for example.

2. Auto-Immune Diseases with Unknown Cause

The presence of an increased level of CD4⁺CD40⁺ T cells (exaggerated level) as compared to the level of cells in a non-autoimmune subject or sample or control population (the standard level) indicates the presence of an auto-immune disease in the subject having the elevated level of CD4⁺CD40⁺ T cells. Thus, the method of the invention can provide a diagnosis of an existing auto-immune disease whether or not the etiology of the auto-immune disease is known.

B. Predictive Tests for Auto-Immune Diseases

1. Predetermined Auto-Immune Diseases

Alternatively, the auto-immune disease may not have yet developed, is inactive, or has not progressed to the point where symptoms or indications are exhibited by the subject, in which case the test i's predictive of expressing the auto-immune disease. The invention also includes a blood test that will predict the susceptibility of an individual towards any predetermined auto-immune disease. This will be accomplished by a blood test kit. In a physician's office, blood samples will be taken. In a laboratory setting, the blood samples will be treated with fluorescent labeled antibodies that recognize the CD4 molecule and antibodies that recognize the CD40 molecule after the sample is contacted with one or more auto-immune disease specific antigens in an amount and for a length of time sufficient to activate the T cells of the predetermined subject. The T cells may be, but are not required to be, in purified or isolated form before contact. Cells that stain positively with both markers will be categorized as “autoaggressive.” While these cells do exist in some low level in normal individuals, they are shown to be increased in “auto-immune” disease prone individuals. Therefore exaggerated levels of CD4⁺CD40⁺ T cells will indicate a propensity to develop auto-immunity. Standard levels or “exaggerated” levels will be determined by establishing a normal level of CD4⁺CD40⁺ T cells in non-auto-immune prone individuals. The levels of CD4⁺CD40⁺ cells are determined using any method appropriate for determining presence or absence of the CD4 and CD40 markers.

Auto-immune diseases for which diagnostic or predictive tests may be established according to the methods of the invention, include but are not limited to, multiple sclerosis (MS), rheumatoid arthritis (RA), systemic lupus erythromatosis, atherosclerosis, Crohn's colitis, ulcerative colitis, primary biliary cirrhosis, chronic obstructive pulmonary disease (COPD) including such as for example, emphysema, allergic asthma and scleroderma, and can be any auto-immune disease for which at least one antigen is known to be involved. For example, type 1 diabetes is known to involve one or more antigens on the surface of pancreatic cells. Similarly, rheumatoid arthritis is known to involve one or more antigens expressed on the surface of synovial tissue; multiple sclerosis is known to involve one or more antigens expressed on the surface of nervous tissue; scleroderma is known to involve one or more antigens expressed on the epidermal or dermal layer of skin tissue; atherosclerosis is known to involve one or more antigens expressed on the surface of cardiac tissue; and, emphysema is known to involve one or more antigens expressed on respiratory tissue and antigens found in tobacco smoke or tobacco products. This invention will characterize the susceptibility of an individual to auto-immune diseases such as type 1 diabetes, rheumatoid arthritis, lupus, atherosclerosis, multiple sclerosis, Crohn's colitis, ulcerative gastritis, primary biliary cirrhosis and auto-immune hepatitis, for example.

For identifying T cells expressing CD4 and CD40, any anti-CD4 or anti-CD40 antibody, or fragment thereof, known in the art may be used. Such antibodies and fragments are commercially available. See, for example, U.S. Pat. No. 5,683,693. Also contemplated for use in the invention are peptides, oligonucleotides or a combination thereof which specifically recognize determinants, such as, for example, CD4 and CD40, with specificity similar to traditionally generated antibodies. See, for example, U.S. Pat. No. 6,365,362.

Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecule. See, for example, U.S. Pat. No. 6,365,362.

II. Methods of Treatment of Auto-Immune Diseases

A. CD40-CD154 Interactions

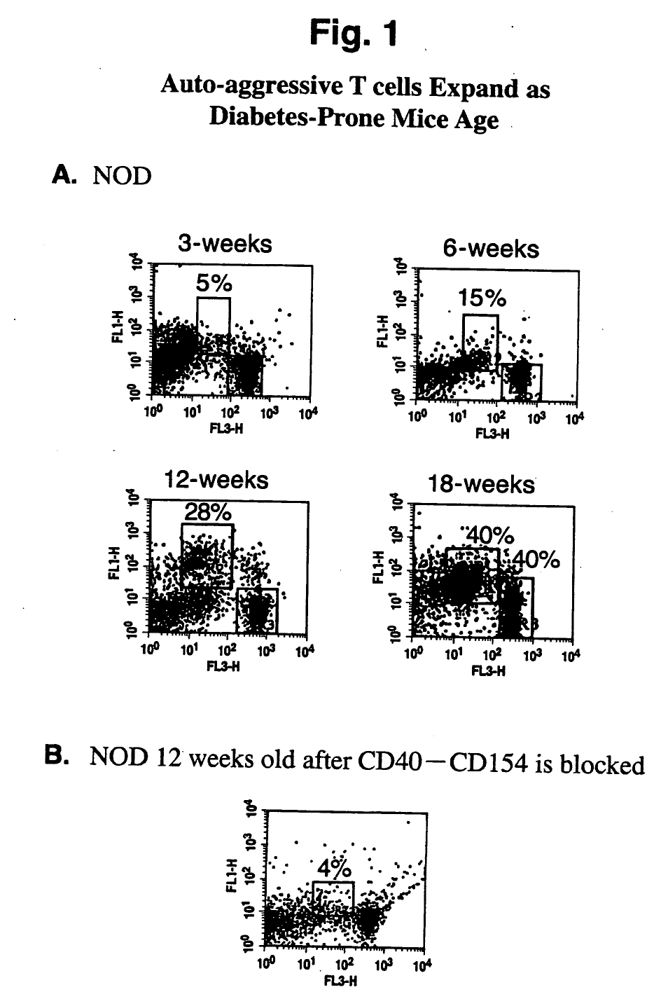

This invention is also related to the use of new drugs or existing drugs to control CD40-CD154 interactions within the auto-aggressive T cell population. Several means of preventing the generation of CD4⁺CD40⁺ auto-aggressive T cells exist. It is possible to treat individuals with an antibody against the CD40 ligand, CD154, or against the CD40 molecule to prevent interaction of those molecules. Preventing this interaction inhibits the development of auto-aggressive T cells (FIG. 1). Another means of preventing CD40 induced activation is to block interaction with CD40 ligand through use of specific peptides (blocking peptides). Because CD40 acts as a “receptor” on auto-aggressive T cells, by designing specific amino acid peptides that can bind to the active site of the CD40 molecule, interaction with the natural ligand for CD40, (CD154) can be prevented. See, for example, U.S. Pat. No. 5,683,693 and Balasa, B. et al. (1997).

Sequence analysis of the CD154 (SEQ ID NO: 6), the natural ligand for CD40, has been determined. From this information inhibiting peptides can be inferred (see, for example, Karpusas, M. et al., Structure 3, 1426 (1995)). Such peptides, include but are not limited to

SSKTTSVLQWAEKGYYTMSNNLVT (SEQ ID NO: 7) and

QIAAHVISEASSK (SEQ ID NO: 8).

The use of blocking peptides will be as follows. We will design peptides that interact with the CD40 antigen. These peptides will not induce the CD40 antigen to activate the T cell. The peptides will prevent interaction of the ligand for CD40, CD40L also known as CD154, with CD40 on the T cells. We have shown that when CD40 is activated on T cells later in life, in a mouse diabetes model, that T cells are induced to alter TCR expression. We predict that this action generates auto-aggressive T cells. By using the blocking peptides we predict that we can successfully prevent the generation of auto-aggressive T cells. Blocking peptides can be used according, for example, to the following protocols.

Protocol #1: Blood samples are taken. The T cells may be purified from the blood sample by standard techniques such as cell sorting or use of anti-CD4 antibodies and purification columns. The blood samples or purified/isolated T cells are incubated with the “blocking peptides.” The blood samples or purified/isolated T cells are then treated with physiological sources of CD40 ligand and assayed for changes in T cell receptor expression such as described in Wagner, D. H., Jr. et al. (2002); Wagner, D. H., Jr. et al., Eur. J. Immunol. 24, 3148 (1994); Wagner, D. H., Jr. et al., J. Exp. Med. 184, 1631 (1996); and Wagner, D. H., Jr. et al. (1999).

Protocol #2: Blocking peptides are administered to patients determined to be at high risk for a specific autoimmune disease, such as assessed using the predictive kit described herein. Blocking peptides are in use therapeutically for several diseases (Lung, F. D. & Tsai, J. Y., Biopolymers 71, 132 (2003); Anderson, M. E. & Siahaan, T. J., Peptides 24, 487 (2003)).

B. RAG Proteins

1. Agents

This invention is also related to the use of new agents or existing agents to control the activation of the RAG proteins within the auto-aggressive T cell population. One means of inhibiting auto-aggressive T cell development is to inhibit the generation of the “self-reactive” T cell receptor. Relative to the RAG1 and RAG2 proteins, there are two ways to control the activity of these proteins. The first is to control the “recombinase” activity of these proteins. Because RAG1 and RAG2 bind to DNA and cut then splice the DNA to generate new TCR molecules, these proteins have a “recombinase” activity (Vaandrager, J. W., et al., Blood 96, 1947-52 (2000)).

Any agent that could prevent this recombination activity potentially would prevent the action of these proteins. Because we have discovered that RAG proteins are exclusively over-expressed in auto-aggressive T cells, agents can be used to inhibit the activation of RAG1 and/or RAG2 genes. Inhibition of RAG activation will inhibit the onset of auto-immune diseases by affecting the generation of auto-aggressive T cells.

Experiment to Show Inhibition of RAG Activity

T cells are isolated using standard techniques such as cell sorter, or T cell-purification columns (Wagner, D. H., Jr. et al. (2002); Vaitaitis, G. M. et al. (2003); Wagner, D. H., Jr. et al. (1994); Wagner, D. H., Jr. et al. (1996); Wagner, D. H., Jr. et al. (1999)). T cells are incubated with different concentrations of 1) integrase inhibitors as described in U.S. Pat. No. 6,403,347 B1; 2) RAG1 and or RAG2 RNAi pools (the RAG RNAi pools are several different combinations of RAG-RNA molecules to maximize efficacy of inhibition); or 3) CD40L blocking peptides. Options 1 and 2 directly inhibit activation of RAGs and option #3 inhibits the CD40 signaling pathways leading to activation of RAGs. Following treatment, T cells will be incubated with agonistic (activating) anti-CD40 antibody, with physiological or nonphysiological sources of CD40L. T cells then will be assayed for changes in T cell receptor molecules. We have shown that anti-CD40 induces changes in T cell receptor expression (Wagner, D. H., Jr. et al. (1999)). Physiological sources of CD40L include activated T cells (Wagner, D. H., Jr. et al., (1994)) and platelets (Andre, P. et al., Circulation 106, 896 (2002); Wang, C. L. et al., Pediatrics 111, E140 (2003)). Nonphysiological sources include isolated, pure or purified preparations of CD40L. T cells that have been treated as in #1, 2 or 3 should not demonstrate changes in TCR expression. As controls, untreated T cells will be treated with anti-CD40 or with CD40L sources and assayed for altered TCR expression. These experiments will determine how blocking CD40-CD154 interaction prevents expansion of altered TCR-bearing T cells. We have determined that T cells that alter TCR expression in the periphery are diabetogenic (Wagner, D. H., Jr. et al., (2002)).

We show that blocking CD40-CD154 interaction inhibits the expansion of auto-aggressive T cells in the type 1 diabetes model (FIG. 1). For physiologic examination, we will treat animals, nonobese mice (NOD)(NOD mice are the accepted animal model for human type 1 diabetes) with integrase inhibitors, such as chaetochromins, using the protocol described in U.S. Pat. No. 6,403,347 B1 or with RNAi molecules or with CD40-blocking peptides (described herein). Animals are closely monitored for expansion of CD4^{lo}CD40⁺ T cells and for diabetes onset.

2. RNAi Molecules

Another important means of preventing RAG1 and or RAG2 activity in auto-immune disease is to prevent the synthesis and accumulation of these proteins within auto-aggressive cells. Because the RAG proteins are synthesized normally in T cells and B cells, it is possible to use a class of drugs inhibitory to the synthesis of these proteins. These drugs include inhibitory RNA (“RNAi”) molecules, specifically designed to inhibit the expression of the RAG1 and RAG2 proteins. RNAi molecules are designed by determining the nucleotide sequence of the RAG1 and RAG2 genes. Such RNAi molecules include but are not limited to

| | |
|---|---|
| 5′-AUGUCUCUGCAGAUGGUAACdAdG-3′; | (SEQ ID NO:9) |
| 5′-CUGUUACCAUCUGCAGAGACdAdU-3′ | (SEQ ID NO:10) |
| 5′-GGUAGGAGAUCUUCCUGAAGdCdC-3′; | (SEQ ID NO:11) |
| 5′-GGGGAUGGGCACUGGGUCCAUGdCdU-3′; | (SEQ ID NO:12) |
| 5′-AGCAUGGACCCAGUGCCCAUCCdCdC-3′; | (SEQ ID NO:13) |
| 5′-CUGUUACCAUCUGCAGAGACdAdU-3′; | (SEQ ID NO:14) |
| 5′-AUGGCAGCCUCUUUCCCACCCAdCdC-3′; | (SEQ ID NO:15) |
| 5′-GGUGGGUGGGAAAGAGGCUGCCdAdU-3′; | (SEQ ID NO:16) |
| 5′-AAACUUGCAGCUCAGCAAAAAACdTdG-3′; | (SEQ ID NO:17) |
| 5′-GAGUUUUUUGCUGAGCUGCAAGUUdUdU-3′; | (SEQ ID NO:18) |
| 5′-GAGUUUUUUGCUGAGCUGCAAGUUdUdU-3′; | (SEQ ID NO:19) |
| 5′-UCACAAAACCCUGGCCCAUGUUdCdC-3′; | (SEQ ID NO:20) |
| and, | |
| 5′-GGAACAUGGGCCAGGGUUUUGUdGdA-3′. | (SEQ ID NO:21) |

When genes are transcribed into messenger RNA that will be translated into protein, a “sense” strand on the gene for that substance is read by the machinery of the cell involved. Small pieces of chemically altered RNA molecules, including but not limited to those above, can be synthesized, that when administered, will go into the cell and bind to the synthesis machinery of that cell to prevent, specifically, the synthesis of the desired protein. This process does not inhibit the synthesis of other proteins within the cell.

This invention also provides kits for the detection and/or quantification of CD4⁺CD40⁺ cells. The kits can include a container containing one or more of any of the above antibodies, antigens or ligands, with or without labels, free, or bound to a solid support as described herein. The kits can also include instructions for the use of one or more of these reagents in any of the assays described herein. For example, antigens envisioned to be useful in the practive of the invention include proteins such as, for example, myosine and actin, and other compounds such as, for example, nicotine and catecholamine. Any protein, biological or nonbiological chemical can conceivably serve as a foreign antigen.

Methods for staining cytokines are standard in the lab. See, for example, Methods of Immunology, Cold Spring Harbor Text book. T cells are isolated from whole blood that is red blood cell depleted, then treated with anti-CD3 or anti-CD3+ anti-CD40 (molecule specific antibodies) for 45 min. Antibodies are washed away in a phosphate buffered saline solution. T cells are incubated in growth media overnight. The media is removed and assayed using enzyme-linked immunosorbant assay (ELISA) specifically for Th1 cytokines, IL-2, IFN-gamma and Th2 cytokines, IL-4, IL-6, and IL-10. For ELISA a plate is coated with antibodies that recognize one of the cytokines of interest. The media is applied and incubated overnight, then the plates are washed. The plates are incubated with a second antibody containing a horseradish peroxidase molecule conjugated to an anti-cytokine antibody, e.g., anti-IL-4 or IL-2, etc. The plate is treated with peroxide and a colorogenic reagent that develops color if the well is positive. The color levels are determined by a spectrophotometer.

A second method is to directly stain T cells for production of cytokines. T cell are treated with anti-CD3 or anti-CD3+ anti-CD40 antibodies in the presence of brefeldin A, a substance that blocks cytokine secretion. T cells are stained on the surface for expression of CD4 and CD40 using appropriate antibodies. T cells are washed and treated with saponin buffer. Saponin is a mild detergent that lyses cells by causing small holes in the cell membrane. The T cells are then incubated with fluorochrome-labeled antibodies, washed and assayed by flow cytometry.

The pharmaceutically acceptable salts of the compounds of this invention include those formed from a variety of cations such as, for example, but not limited to, sodium, potassium, aluminum, calcium, lithium, magnesium, zinc, and from bases such as ammonia, ethylenediamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, e.g. by reacting the free acid with a suitable organic or inorganic base. Many other suitable cations and bases are known in the art, see, for example, Remington's, and U.S. Pat. No. 6,403,347, and are envisioned in the practice of the invention.

For modulating the proliferation of the CD4^{lo}CD40^{hi} lymphocytes, the agents of the present invention may be administered by a variety of routes, including, but not limited to, orally, as subcutaneous injections, by intravenous, intramuscular, intrasternal injection or infusion techniques, by inhalation spray, topically, or rectally, such as in suppositories, in dosage unit formulations containing conventional non-toxic pharmaceutically-acceptable carriers, adjuvants and vehicles.

Thus, in accordance with the present invention the contacting involves contacting a subject in need of such treatment with a composition comprising a pharmaceutical carrier and a therapeutically-effective amount of at least one agent of the present invention. The compositions may be in variety of orally-administrable forms, such as but not limited to, suspensions or tablets, nasal sprays, sterile injectible preparations, for example, as sterile injectible aqueous or nonaqueous suspensions. See, for example, U.S. Pat. No. 6,403,347 and Remington's.

When administered orally, these compositions are prepared according to techniques well-known in the art of pharmaceutical formulation and may contain, by way of example, microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art. See, for example, U.S. Pat. No. 6,403,347 and Remington's.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,403,347 and Remington's.

The injectible solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. When rectally administered in the form of suppositories, these compositions may be prepared by mixing the agent with a suitable non-initiating excipient, such as cocoa butter, synthetic glyceride esters of polyethylene glycols, which are solid at ordinary temperatures, but liquefy and/or dissolve in the rectal cavity to release the drug. See, for example, U.S. Pat. No. 6,403,347 and Remington's.

The agents of the present invention can be administered orally to humans or other mammals in a dosage range of 1 to 1000 mg/kg body weight in divided doses. One preferred dosage range is 0.1 to 200 mg/kg body weight orally in divided doses. Another preferred dosage range is 0.5 to 100 mg/kg body weight orally in divided doses. For oral administration, the agents are preferably provided in the form of tablets containing 1.0 to 1000 milligrams of the active ingredient, particularly in 0.001, 0.01, 0.1, 0.5 or 1.0 milligram increments, for the symptomatic adjustment of the dosage to the subject to be treated. It will be understood, however, that the specific dose level and frequency of dosage for any particular subject may be varied and will depend upon a variety of factors including the activity of the specific agent employed, the metabolic stability and length of action of that compound, the age, body weight, general health, sex, diet, mode and time of administration, rate of excretion, drug combination, the severity of the particular condition, and the subject in need of having the proliferation of CD4^{lo}CD40^{hi} lymphocytes modulated. See, for example, U.S. Pat. Nos. 6,403,347; 6,110,716; 5,683,693 and Remingtons's.

Also envisioned in the practice of the invention is a composition comprising a combination of at least two of the following: a combination comprising one or more agent which inhibits the activation of RAG recombinase; an antibody molecule or fragment thereof to CD40; an antibody molecule of fragment thereof to CD154; at least one blocking peptide which inhibits the interaction of the CD40 receptor with the CD154 ligand; at least one RNA molecule specifically hybridizing to the RAG2 gene product; and, at least one RNA molecule specifically hybridizing to the RAG1 gene product.

The following examples are provided to facilitate the practice of the present invention. These examples are not intended to limit the scope of the invention in any way.

III. Differentiation of Type 1 from Type 2 Diabetes

Autoimmune diseases are characterized by aseptic, chronic inflammation leading to debilitating tissue destruction. In type 1 diabetes (T1D), while several cell types participate in the inflammatory process, T cells infiltrate the pancreatic islets and attack the insulin-producing β cells thus affecting insulin production leading to hyperglycemia (Wagner, D. H., Jr., Vaitaitis, G., Sanderson, R., Poulin, M., Dobbs, C. & Haskins, K. (2002) Proc Natl Acad Sci USA 99, 3782-7; Vaitaitis, G. M., Poulin, M., Sanderson, R. J., Haskins, K. J. & Wagner Jr., D. H. (2003) Cutting Edge, J. Immunol. 170, 3455-3459; Waid, D. M., Vaitaitis, G. M. & Wagner, J., D. H. (2004) European Journal of Immunology 34, 1488-1497; Bach, J. F. & Chatenoud, L. (2001) Annu Rev Immunol 19, 131-61). Furthermore, the disease causes complications including, diabetic retinopathy, neuropathy, nephropathy, digestive and respiratory complications and atherosclerotic lesions among others (Mokuno, T., Sawai, Y., Oda, N., Mano, T., Hayakawa, N., Kato, R., Itoh, Y., Shimazaki, K., Kotake, M., Nakai, A., Hiramitsu, S., Itoh, M., Morimoto, S. & Nagasaka, A. (1996) Diabetes Care 19, 374-8; Reaven, G. M., Thompson, L. W., Nahum, D. & Haskins, E. (1990) Diabetes Care 13, 16-21).

In the nonobese diabetic (NOD) mouse T1D model we identified a unique sub-population of T cells characterized as CD4⁺CD40⁺ that proved to be highly auto-aggressive (Id.). Occurring at low levels in young mice these T cells increase substantially (up to 56% of the CD4⁺ compartment), and concurrently with the progressive insulitis as mice develop T1D (Id.). While detected in non-autoimmune animals, CD4⁺CD40⁺ T cells consistently are contained at low levels in those strains (Id.). Pathogenicity of CD4⁺CD40⁺ T cells was confirmed through adoptive transfers to NOD.scid recipients. In those experiments, the CD4⁺CD40⁺ T cells from either diabetic or pre-diabetic animals but not CD4⁺CD40⁻ T cells infiltrated the pancreatic islets causing depletion of insulin resulting in hyperglycemia (Id.). This demonstrates pathogenicity and that the auto-aggressor T cell subset precedes clinical onset of diabetes.

A recent study demonstrated that human T cell clones, including insulin responsive T cells, could be generated from pancreatic lymph nodes of diabetic patients (Kent, S. C., Chen, Y., Bregoli, L., Clemmings, S. M., Kenyon, N. S., Ricordi, C., Hering, B. J. & Hafler, D. A. (2005) Nature 435, 224-8). The majority of T cells expressed a restricted TCR repertoire, specifically Vα8⁺ or Vα39⁺ in pancreatic nodes of T1D patients and were HLA-DR4 restricted (Kent, S. C., Chen, Y., Bregoli, L., Clemmings, S. M., Kenyon, N. S., Ricordi, C., Hering, B. J. & Hafler, D. A. (2005) Nature 435, 224-8). This data strongly suggest that a limited repertoire of T cells may prove causal in T1D.

The observation that the CD4^{lo}CD40⁺ effector T cell subset is highly diabetogenic in NOD mice led to possibility that these T cells may prove to have a corollary population in human T1D and that it may be possible to detect this subset in peripheral blood. The levels of CD4⁺CD40⁺ T cells were compared in diagnosed T1 D and control patients and compared in T1D and type 2 diabetes (T2D) patients. While these diseases have obviously similar clinical manifestations, T1D is classically autoimmune and T2D is not. By identifying and focusing on a unique T cell subset that is directly causal for diabetes as demonstrated in the NOD model, it may possible to distinguish type 1 from type 2 diabetes through a simple blood test. Most importantly, these T cells are not expanded in T2D patients, demonstrating a diagnostic difference between T1 and T2 diabetes.

Accordingly, the invention herein provides a method for differentiating whether a test subject has type-1 or type-2 diabetes by a) obtaining a blood sample from the test subject; b) detecting CD4⁺CD40⁺ T cells in the blood sample; and c) determining whether the CD4⁺CD40⁺ T cells in the blood sample are expanded by comparing to CD4⁺CD40⁺ T cells in a control sample of blood, wherein an expanded level of CD4⁺CD40⁺ T cells indicate that the subject has type-1 diabetes and not type-2 diabetes.

In some embodiments, the blood comprises peripherial blood. In one embodiment, the blood sample consists essentially of peripheral blood. Peripherial blood can be obtained by a number of standard laboratory procedures well known to those of skill in the art.

T cells are isolated from blood using standard techniques such as cell sorter, or T cell-purification columns (Wagner, D. H., Jr. et al. (2002); Vaitaitis, G. M. et al. (2003); Wagner, D. H., Jr. et al. (1994); Wagner, D. H., Jr. et al. (1996); Wagner, D. H., Jr. et al. (1999)). For identifying T cells expressing CD4 and CD40, any anti-CD4 or anti-CD40 antibody, or fragment thereof, known in the art may be used. Such antibodies and fragments are commercially available. See, for example, U.S. Pat. No. 5,683,693, which is herein incorporated by reference in its entirity. Also contemplated for use in the invention are peptides, oligonucleotides or a combination thereof which specifically recognize determinants, such as, for example, CD4 and CD40, with specificity similar to traditionally generated antibodies. See, for example, U.S. Pat. No. 6,365,362, which is herein incorporated by reference in its entirity.

Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecule. See, for example, U.S. Pat. No. 6,365,362, which is herein incorporated by reference in its entirity. In some embodiments of the invention, the subject is a mammal. In one embodiment, the subject is a human.

In some embodiments, the control sample of blood is from a subject not diagnosed with type-1 or type-2 diabetes. In other embodiments, the control sample of blood is from a subject with type-2 diabetes. The control sample of blood may be from a healthy subject or a subject without any autoimmune disease.

A number of methodologies may be employed to quantitate the number of CD4⁺CD40⁺ T cells for determining whether a subject has an auto-immune disease and for distinguishing whether a subject has type 1 or type 2 diabetes. Those skilled in the art will appreciate that the methods indicated below represent some of the perferred ways in which the number of CD4⁺CD40⁺ T cells can be determined and in no manner limit the scope of methodolgies that may be employed. Those skilled in the art will be able to determine operative and optimal assay conditions for each determination by employing routine experimentation. As examples, CD4⁺CD40⁺ T cells can be quantitated according to the techniques below.

Flow Cytometry. In one embodiment of the invention, flow cytometry can be used to quantitate the number of CD4⁺CD40⁺ T cells present within a blood sample. Flow cytometry objectively quantifies and separates single cells on the basis of one or more parameters (e.g., binding to a pre-selected antigen). Commercially available flow cytometers may be used. Manufacturers of such devices include Coulter, Becton-Dickenson, and Cytomation.

As applied to the method of this invention, the flow cytometer would have a selection of lasers which provide light to match the excitation frequency of the particular compound used to produce fluorescence in the cell sample. By this fluorescent tagging, the flow cytometer is able to accurately count the number of cells which fluoresce. Flow cytometry involves channeling individual cells in a narrow fluid stream past a laser beam, which is usually oriented at a right angle to the flow. Optical sensors detect signals generated as the cells pass through the laser beam. The cells scatter the laser light in proportion to their size and “complexity” (e.g. presence of granules in their cytoplasm). Thus, cells can be identified based on their light scatter characteristics, and a population chosen (gated) for further analysis.

In some embodiments, pre-selected antigens (CD4 and CD40) may be coupled to fluorochromes (different fluorochromes emit different wavelengths of light upon excitation by a laser) are used to label or “stain” the cells so that each cell can be identified and quantitated based upon its fluorescence signal.

In other embodiments, secondary antibodies that specifically bind to the pre-selected antigen (for example, a secondary antibody may be used to bind to anti-CD4 antibody, which detected the original antigen, CD4) are coupled to fluorochromes and used for detection. A computer collects the fluorescence signature of each cell and displays the pattern of fluorescence for the user to analyze.

In further embodiments, where one might want to separate cells which have a certain staining pattern from all other cells (e.g., due to binding to a labeled pre-selected antigen), the flow cytometry machine can direct those desired cells into a tube provided by the user. This is called fluorescence activated cell sorting (“FACS”). FACS involves the inducement of a charge (positive or negative) on the cell surface of each cell which passes through the flow cytometer. By this induced charge, the fluorescing cells are separated from non-fluorescing cells. Accordingly, the fluorescing cells would be placed into a separate container from the non-fluorescing cells. However, it shall be understood that the method of this invention is not limited to any particular sequence in terms of using a flow cytometer or conducting a FACS.

Enzyme-Linked Immunosorbent Assay (“ELISA”). In one embodiment of the invention, ELISA can be used to quantitate the number of CD4+CD40+ T cells present within a blood sample. Many ELISA applications and formats have been described. Various sources provide discussion of ELISA chemistry, applications, and detailed protocols (See e.g., Crowther, “Enzyme-Linked Immunosorbent Assay (ELISA),” in Molecular Biomethods Handbook, Rapley et al., pp. 595-617, Humana Press, Inc., Totowa, N.J. (1998); Harlow and Lane (eds.), Antibodies. A Laboratory Manual, Cold Spring Harbor Laboratory Press (1988); Ausubel et al. (eds.), Current Protocols in Molecular Biology, Ch. 11, John Wiley & Sons, Inc., New York (1994); and Laurino et al., Ann. Clin. Lab Sci., 29(3):158-166 (1999)).

In preferred embodiments of the present invention, ELISA methods for quantitation of antigen are provided. In some of these methods, the antigen (e.g., CD4⁺ and CD40⁺ T cells) are first immobilized on a solid support (e.g. in a microtiter plate well). Detection and quantitation of the immobilized antigen is accomplished by use of an antibody-enzyme conjugates capable of binding to CD4 and CD40 and producing a quantifiable signal. In some embodiments, the amount of antigen present is directly proportional to the amount of enzyme reaction product produced after the addition of an appropriate enzyme substrate.

As indicated previously, enzymes commonly used in ELISAs include horseradish peroxidase (HRPO), urease, alkaline phosphatase, glucoamylase and P-galactosidase. Protocols for the preparation of suitable antibody-enzyme conjugates are well known in the art. The present invention provides methods for the preparation of an antibody-enzyme (i.e., HRPO enzyme) conjugate that specifically recognizes the antigens of interest (i.e., CD4 and CD40) for use in an immunoassay (e.g., ELISA). As those of skill in the art will recognize other methods for antibody-enzyme conjugation may be used with the present invention.

Conjugation of enzymes to antibodies involves the formation of a stable, covalent linkage between an enzyme (e.g., HRPO or alkaline phosphatase) and the antibody (e.g., the anti-CD4 and anti-CD40 antibodies), where neither the antigen-binding site of the antibody nor the active site of the enzyme is functionally altered.

The conjugation of antibody and HRPO is dependent on the generation of aldehyde groups by periodate oxidation of the carbohydrate moieties on HRPO (Nakane and Kawaoi, J. Histochem. Cytochem., 22:1084-1091 (1988)). Combination of these active aldehydes with amino groups on the antibody forms Schiff bases that, upon reduction by sodium borohydride, become stable.

Protocols to make antibody-enzyme conjugates using urease or alkaline phosphatase enzymes are also known in the art (Healey et al., Clin. Chim. Acta 134:51-58 (1983); Voller et al., Bull. W. H. O., 53:55-65 (1976); and Jeanson et al., J. Immunol. Methods 111:261-270 (1988)). For urease conjugation, cross-linking of the urease enzyme (e.g., Urease Type VII, Sigma No. U0376) and antibody using m-maleimidobenzoyl N-hydroxysuccinimide ester (MBS) is achieved through benzoylation of free amino groups on the antibody. This is followed by thiolation of the maleimide moiety of MBS by the cysteine sulfhydryl groups of urease. To prepare an alkaline phosphatase-antibody conjugate, a one-step glutaraldehyde method is the simplest procedure (Voller et al., Bull. W. H. O., 53:55-65 (1976)). This antibody-alkaline phosphatase conjugation protocol uses an enzyme immunoassay grade of the alkaline phosphatase enzyme.

The end product of an ELISA is a signal typically observed as the development of color or fluorescence. Typically, this signal is read (i.e., quantitated) using a suitable spectrocolorimeter (i.e., a spectrophotometer) or spectrofluorometer. The amount of color or fluorescence is directly proportional to the amount of immobilized antigen. In some embodiments of the present invention, the amount of antigen in a sample (e.g., the amount of CD4 and CD40) is quantitated by comparing results obtained for the sample with a series of control wells containing known concentrations of the antigen (i.e., a standard concentration curve). A negative control is also included in the assay system.

It is contemplated that any suitable chromogenic or fluorogenic substrates will find use with the enzyme-conjugated antibodies of the present invention. In some embodiments of the present invention, the substrate p-nitrophenyl phosphate (NPP) in diethanolamine is the preferred substrate for use in colorimetric ELISA methods, and 4-methylumbelliferyl phosphate (MUP) is the preferred alkaline phosphatase substrate in fluorometric ELISA methods. Conjugated antibodies can include radioisotopes, fluorophores, enzymes, luminescers, or visible particles (e.g., colloidal gold and dye particles). These and other labels are well known in the art and are described, for example, in the following U.S. Pat. Nos. 3,766,162; 3,791,932; 3,817,837; 3,996,345; and 4,233,402, which are hereby incorporated by reference in their entirety.

The present invention provides various ELISA protocols for the detection and/or quantitation of CD4⁺CD40⁺ T cells in a sample. In one embodiment, the present invention provides a “direct ELISA” for the detection of CD4⁺CD40⁺ T cells in a sample. In some embodiments, the antigen of interest in a sample (i.e., the CD4⁺CD40⁺ T cells) are bound (along with unrelated antigens) to the solid support (e.g., a microtiter plate well). The immobilized antigen is then directly detected by the antigen-specific enzyme-conjugated antibody, also provided by the present invention. Addition of an appropriate detection substrate results in color development or fluorescence that is proportional to the amount of CD4⁺CD40⁺ T cells present in the well.

In another embodiment, the present invention provides an indirect ELISA for the detection of CD4⁺CD40⁺ T cells in a sample. In this embodiment, antigen of interest in a sample is immobilized (along with unrelated antigens) to a solid support (e.g., a microtiter plate well) as in the direct ELISA, but is detected indirectly by first adding an antigen-specific antibody, then followed by the addition of a detection antibody specific for the antibody that specifically binds the antigen, also known as “species-specific” antibodies (e.g., a goat anti-rabbit antibody), which are available from various manufacturers known to one in the art (e.g., Santa Cruz Biotechnology; Zymed; and Pharmingen/Transduction Laboratories).

In another embodiment, the present invention provides “sandwich ELISA” methods, in which the antigen in a sample is immobilized on a solid support by a “capture antibody” that has been previously bound to the solid support. In general, the sandwich ELISA method is more sensitive than other configurations, and is capable of detecting 0.1-1.0 ng/ml protein antigen. As indicated above, the sandwich ELISA method involves pre-binding the “capture antibody” which recognizes the antigen of interest (i.e., CD4⁺CD40⁺ T cells) to the solid support (e.g., wells of the microtiter plate). In some embodiments, a biotinylated capture antibody is used in conjunction with avidin-coated wells. Test samples and controls are then added to the wells containing the capture antibody. If antigen is present in the samples and/or controls, it is bound by the capture antibody.

In some embodiments, after a washing step, detection of antigen that has been immobilized by the capture antibody is detected directly (i.e., a direct sandwich ELISA). In other embodiments detection CD4⁺CD40⁺ T cells that have been immobilized by the capture antibody is detected indirectly (i.e., an indirect sandwich ELISA). In the direct sandwich ELISA, CD4⁺CD40⁺ T cells are detected using an antigen-specific enzyme-conjugated antibody. In the indirect sandwich ELISA, the captured CD4⁺CD40⁺ T cells (i.e. antigen) are detected by using an antibody directed against the antigen, which is then detected by another enzyme-conjugated antibody which binds the antigen-specific antibody, thus forming an antibody-antigen-antibody-antibody complex. In both the direct and indirect sandwich ELISAs, addition of a suitable detection substrate results in color development or fluorescence that is proportional to the amount of antigen that is present in the well.

In the sandwich ELISA, the capture antibody used is typically different from the second antibody (the “detection antibody”). The choice of the capture antibody is empirical, as some pairwise combinations of capture antibody and detection antibody are more or less effective than other combinations. The same monoclonal antibody must not be used as both the capture antibody and the conjugated detection antibody, since recognition of a single epitope by the capture antibody will preclude the enzyme-conjugated detection antibody from binding to the antigen. However, in some embodiments, two different monoclonal antibodies that recognize different epitopes are used in this assay. In other embodiments, the same polyclonal antibody preparation is used as both the capture antibody and conjugated detection antibody, since multiple epitopes are recognized in the pool of polyclonal antibody species. In some embodiments, the polyclonal anti-CD4 and anti CD40 antibodies provided by the present invention is used as both the capture antibody and the enzyme-conjugated detection antibody in the sandwich ELISA.

Furthermore, it is not intended that the present invention be limited to the direct ELISA and sandwich ELISA protocols particularly described herein, as the art knows well numerous alternative ELISA protocols that also find use in the present invention (See, e.g., Crowther, “Enzyme-Linked Immunosorbent Assay (ELISA),” in Molecular Biomethods Handbook, Rapley et al., pp. 595-617, Humana Press, Inc., Totowa, N.J. (1998); and Ausubel et al. (eds.), Current Protocols in Molecular Biology, Ch. 11, John Wiley & Sons, Inc., New York (1994)). Thus, any suitable ELISA method including, but not limited to, competitive ELISAs also find use with the present invention.

The present invention also provides ELISA amplification systems. These embodiments produce at least 10-fold, and more preferably, a 500-fold increase in sensitivity over traditional alkaline phosphatase-based ELISAs. In one preferred embodiment of the ELISA amplification protocol, bound alkaline phosphatase acts on an NADPH substrate, whose reaction product initiates a secondary enzymatic reaction resulting in a colored product. Each reaction product from the first reaction initiates many cycles of the second reaction in order to amplify the signal (See e.g., Bio-Rad ELISA Amplification System, Cat. No. 19589-019).

Quantitative RT-PCR and Northern Blot Analysis. In yet another embodiment, the number of CD4⁺CD40⁺ T cells can be assayed by a real time quantitative RT-PCR based method. Those of skill in the art will appreciate that many variations of the technique are possible.

The primers used for PCR are suitably designed to comprise nucleotide sequences which encode amino acid sequences that are highly conserved within both the CD4 and CD40 genes. Methods to identify nucleotide sequences corresponding to a given amino acid sequence include deduction on the basis of the codon usage of the host cell, and methods of making mixed oligonucleotide sequences using multiple codons (hereinafter referred to as a ‘degenerate oligonucleotides’). In the latter case, the multiplicity of oligonucleotides can be reduced by introducing hypoxanthine to their nucleotide sequences.

Primers for PCR amplification of the CD4 CD40 genes may comprise a nucleotide sequence designed to anneal with a template chain, the primer being joined to an additional 5′ sequence. The choice of such an additional 5′ nucleotide sequence is not particularly limited, as long as the primer can be used for PCR or RT-PCR. Such an additional 5′ sequence can be, for example, a nucleotide sequence convenient for the cloning operation of a PCR product. Such a nucleotide sequence can be, for example, a restriction enzyme cleavage site or a nucleotide sequence containing a restriction enzyme cleavage site.

Furthermore, in designing of the primer for PCR it is preferred that the sum of the number of guanine (G) and the number of cytosine (C) bases is 40 to 60% of the total number of bases. Furthermore, there is little or no self-annealing for a given primer and, in the case of a pair of primers, little or no annealing between the primers.

The number of nucleotides making up the primer for PCR amplificartion of the CD4 and CD40 genes is not particularly limited, as long as it can be used for PCR. The lower limit of the number is generally 10 to 14 nucleotides, with the upper limit 40 to 60 nucleotides. The primers may be 14 to 40 oligonucleotides in length.

The primers for PCR amplification of the CD4 and CD40 genes are preferably DNA. Nucleosides in the primer can be deoxy adenosine, deoxy cytidine, deoxy thymidine, and deoxy guanosine, and additionally deoxy inosine. The 5′-position of the nucleoside at the 5′-end of the primer for PCR is suitably a hydroxyl group or a hydroxy group to which one phosphoric acid is bonded by an ester link.

Synthesis of primer for PCR amplification of the CD4 and CD40 genes can be performed by methods generally used for synthesis of nucleic acids, for example, the phosphoamidite method. An automated DNA synthesizer can be preferably used in such a method.

Genomic DNA and mRNA from whole blood, or a T cell population can be used as a template for PCR or RT-PCR respectively. Total RNA can also be used as a template for RT-PCR instead of mRNA.

Relative quantitation of CD4 and CD40 mRNA is achieved by means of the ABI Prism 7700 Sequence Detection System (Applied Biosystems, Foster City, Calif.). In TaqMan real-time quantitation technology, the 5′exonuclease activity of the Taq polymerase cleaves and releases a hybridization probe that is labeled with a fluorescent reporter dye. This fluorogenic probe is specific for the target sequence, thereby generating a fluorescence signal that is specific and is directly proportional to the amount of PCR product synthesized. PCR reactions are characterized by the time-point during cycling when amplification of the PCR product is first detected, rather than the amount of product accumulated after a fixed number of cycles. Since the amount of product at the exponential phase of the PCR is proportional to the initial copy number of the target, the more abundant the starting quantity of a target, the earlier will the PCR amplification be detected by means of the fluorescence signal. In this technology, the target quantity is measured by identifying the threshold cycle number (C_{T}), i.e. when the fluorescence signal crosses a preset detection threshold. The laser detector of the Prism 7700 monitors the cycle to cycle change in fluorescence signal on-line. The fewer cycles it takes to reach a detectable level of fluorescence, the greater the initial copy number.

In yet another embodiment of the invention, the number of cells expressing CD4 and CD40 is determined by methods which detect particular mRNAs in cells. These include, hybridization assays using complementary DNA probes (such as in situ hybridization using labeled CD4 and/or CD40 riboprobes, Northern blot and related techniques) and various nucleic acid amplification assays (such as RT-PCR using complementary primers specific for CD4 and/or CD40, and other amplification type detection methods, such as, for example, branched DNA, SISBA, TMA and the like). Protocols for the detection of specific mRNAs in a sample are well known in the art (Sambrook et al., (1990) Molecular Cloning—A Laboratory Manual, Cold Spring Harbor Laboratory Press; Ausubel et al., (1998) Current Protocols in Molecular Biology, Wiley).

Moreover, the present invention provides a simple blood test to determine whether a hyperglycemic individual has the autoimmune form (type 1) of diabetes or the non-autoimmune form (type 2). The present invention also provides a kit for performing the blood test, as an example, blood samples may be taken from the subject. The blood samples may be treated with fluorescent labeled antibodies that recognize the CD4 molecule and antibodies that recognize the CD40 molecule. The T cells may be, but are not required to be, in purified or isolated form before contact. Cells that stain positively with both markers will be quantiated. While these cells do exist in some low level in normal individuals, they are shown to be increased in “auto-immune” disease prone individuals. Therefore exaggerated or expanded levels of CD4⁺CD40⁺ T cells will indicate that the hyperglycemic individual has the autoimmune form of diabetes. Standard levels or “exaggerated” levels may be determined by establishing a normal level of CD4⁺CD40⁺ T cells in non-auto-immune prone individuals or individuals with type 2 diabetes. The levels of CD4⁺CD40⁺ cells are determined using any method appropriate for determining presence or absence of the CD4 and CD40 markers.

Fluorscence In Situ Hybridization. In yet another embodiment, CD4⁺CD40⁺ T cells can be detected by fluorscence in situ hybridization (“FISH”). The term “in situ hybridization” generally refers to hybridization of a nucleic acid probe to a nucleic acid target that is part of a cytological or histological preparation. Those of skill in the art will appreciate that many variations of the technique are possible.

A FISH based method to identify CD4⁺CD40⁺ T cells may comprise the following steps: (a) fixing the tissue or other biological material under investigation to a support (e.g., glass slide or wall of a micro titer well), (b) treatment of the tissue or material to increase accessibility of probe DNA to target DNA, (c) contacting the tissue or material containing the target DNA with probes (specific for CD4 and CD40) to form specific hybridization complexes, (d) post hybridization washes of the complexes to selectively remove probes that are not specifically hybridized to the target, and (e) detection of CD4 and CD40 probes that have formed hybridization complexes with target DNA molecules. Such methods are described in a number of sources, including: Gall and Pardue, (1981) Methods of Enzymology 21:470-480; Henderson, (1982) International Review of Cytology, 76:1-46; and Angerer, et al., (1985) in Genetic Engineering: Principles and Methods (Setlow and Hollaender, Eds.) vol. 7, pp. 43-65, Plenum Press, New York. CD4⁺CD40⁺ T cells identified through FISH, may be quantiated by any known method in the art.

This invention is further illustrated by the following examples which are provided to facilitate the practice of the present invention. These examples are not intended to limit the scope of the invention in any way.

### EXAMPLES

### Example 1

Materials and Methods.

Mice. Nonobese diabetic (NOD), Nonobese resistant (NOR) and BALB/c mice were purchased from Jackson Laboratories, Bar Harbor, Me.; bred and maintained under pathogen-free conditions in the IUCAC approved animal facility at the Webb-Waring Institute, University of Colorado Health Sciences Center, Denver, Colo.

Staining. T cells were purified from excised spleens of NOD, NOR or BALB/c mice at the ages indicated, incubated on nylon wool wetted columns with HBSS-5% BSA for 45 min. Purified T cells (>92% CD3⁺) were washed with HBSS-5% BSA, treated with 2.4.G2, anti-Fc-receptor blocking antibody, then stained with directly conjugated FITC-anti-CD40, 1C10³⁷, PE-anti-TCRαβ, H57.597 or PE-anti-CD3, 145.2C11 (Pharmingen, San Diego, Calif.), and CyChrome™-anti-CD4, H129.19 (Pharmingen). Cells were run on a Becton-Dickinson FACScalibur and assayed using CeIlQuest™ software. In some cases, splenic T cells were incubated with biotin-anti-CD3 (145.2C11), washed with HBSS, incubated with Miltenyi (Auburn, Calif.) magnetic avidin beads and passed through a Miltenyi selection column as per manufacturer's instructions. Purified T cells were then stained as described.

For Vα staining, purified T cells were left untreated or crosslinked with biotin anti-CD40 followed by avidin for 18 hr. T cells were incubated with 2.4.G2, then stained with FITC anti-Vα2, anti-Vα3.2 or anti-Vα8.3 (all from Pharmingen), biotinylated anti-CD40 (1C10) with PE-avidin (Pharmingen), and CyChrome-anti-CD4 (Pharmingen) for analysis.

Adoptive Transfers. T cells were nylon wool-purified from spleens of diabetic and pre-diabetic NOD females, incubated with biotinylated anti-CD40 (1C10 produced in-house), biotinylated anti-Vα3.2, or biotinylated anti-Vα8.3 (both from Pharmingen). The cells were washed with PBS then incubated with magnetic avidin beads (Miltenyi, Auburn, Calif.) and passed over magnetic purification columns (Miltenyi). Purified T cells were eluted and determined to be >98% pure by flow cytometry. CD8+ T cells were removed by incubating T cells with a magnetic conjugated anti-CD8 antibody (Miltenyi) then passed over a magnetic column (Miltenyi). Purified CD4⁺CD40⁺ T cells, 1.5×10⁶, were injected intraperitoneally, i.p., into 9-day old NOD.scid recipients. Control animals received CD4⁺CD40⁻ T cells, 1.5×10⁶ cells. Animals were monitored for diabetes onset by blood glucose (b.g.) determinations. Diabetes was considered to be a b.g. level of >150 mg/dl.

Highly purified Vα3.2⁺ and Vα8.3⁺ T cells, 1.5×10⁶, were injected i.p. into 9-day old NOD.scid recipients that were monitored for diabetes as before. Controls received an equivalent number of CD40⁻ T cells. Vα3.2⁺ T cells were determined to be >80% CD40⁺ while Vα8.3⁺ T cells were <30% CD40⁺. Experiments were repeated three times.

Histology. Pancreata from CD4⁺CD40⁺ and from CD4⁺CD40⁻ T cell NOD.scid recipients were fixed in formalin, paraffin embedded, and sliced by microtome to generate tissue slides. Slides were stained with Hematoxylin and Eosin (H&E) or Aldehyde Fuchsin (A/F) as described previously (Wagner, D. H., Jr. et al., (2002)). Slides were scored for infiltration and insulin production as described (Wagner, D. H., Jr. et al., (2002)).

Specific TCRVα⁺ Expansions within the CD4^{lo}CD40⁺ Auto-Aggressive T Cell Population Promote Type 1 Diabetes

The current study herein demonstrates that CD4⁺CD40⁺ T cells, including for the first time T cells purified from pre-diabetic animals, rapidly transfer diabetes to NOD.scid recipients. Importantly, these T cells expand as NOD mice develop diabetes. Furthermore, there are CD40 driven expansions of TCR Vα3.2⁺ and Vα8.3⁺ T cells within the auto-aggressive T cell population but these expansions are confined to the auto-immune strain. In addition this study shows that primary CD40⁺Vα3.2⁺ T cells induce diabetes with the same kinetics as established diabetogenic T cell clones while Vα8.3⁺ T cells do not induce diabetes. The data presented herein show that specific Vα⁺ T cells are predictive of diabetes onset. All mammals specifically humans demonstrate CD4^{lo}CD40⁺ T cells.

Introduction

Numerous cell types are involved in the development of auto-immune diseases including type 1 diabetes (T1D). Auto-aggressive T cells though are fundamental in progression of the disease (Wagner, D. H., Jr. et al., (2002); Mathis, D. et al., Nature 414, 792-8 (2001); Candeias, S. et al., Proc. Natl. Acad. Sci. USA 88, 6167-70 (1991); Dilts, S. M. et al., J. Autoimmun 13, 285-90 (1999); Haskins, K. & Wegmann, D. (1996); Katz, J. D. et al., Cell 74, 1089-100 (1993)). Studies involving adoptive transfers of diabetogenic T cell clones to nonobese diabetic (NOD) mice and studies using diabetogenic-TCR, transgenic (TCR-Tg) mice demonstrate that CD4⁺ T cells infiltrate the pancreatic β cells leading to loss of insulin production (Candeias, S. et al., (1991); Haskins, K. & Wegmann, D. (1996)). CD8⁺ TCR-Tg NOD mice develop diabetes suggesting a role for CD8⁺ T cells in disease progression (Amrani, A. et al., Immunity 16, 719-32 (2002)). However, when primary CD8⁺ T cells are used, CD4⁺ T cell help is required to fulminate disease (Lejon, K. & Fathman, C. G., J. Immunol. 163, 5708-5714 (1999)).

While diabetogenic T cell clones and TCR-Tg mice provide information about the disease process, it is important to address primary T cells as disease culprits. Recently we suggested that auto-aggressive T cells in the NOD arise from a peripheral subset of T cells that express CD40 (Wagner, D. H., Jr. et al., (2002)). Further studies demonstrate that these T cells are induced through CD40 to transcribe, translate and translocate the recombinase RAG1 and RAG2 proteins to the nucleus (Vaitaitis, G. M. et al., (2003)). Because RAGs function to alter TCR expression, this suggests that CD40 signals contribute to altered TCR expression post thymic selection; perhaps leading to the generation of auto-aggressive T cells in the periphery as opposed to escape from thymic negative selection.

Results

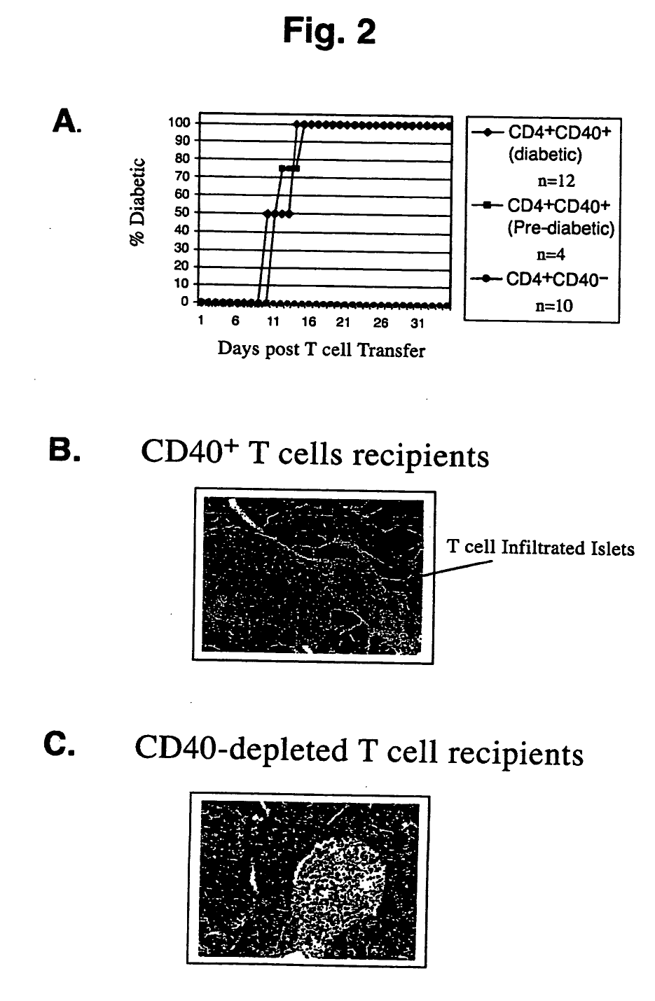

Purified CD4⁺CD40⁺ T cells Are Highly Diabetogenic. We demonstrated previously that a subset of T helper cells in NOD mice characterized as CD4^{lo} successfully transfers T1D (Wagner, D. H., Jr. et al., (2002)). However, substantial numbers (2×10⁷) and multiple injections of these T cells were required to achieve diabetes. Here we demonstrate directly, through use of highly purified CD4⁺CD40⁺ T cells, that relatively low numbers, 1.5×10⁶, of cells rapidly induced diabetes (FIG. 2A). Importantly, highly purified CD4^{lo}CD40⁺ T cells isolated from 9-week old, pre-diabetic NOD animals could successfully transfer diabetes (FIG. 2A). Previous reports suggest that only T cells from diabetic NOD mice can successfully transfer diabetes (Christianson, S. W. et al., Diabetes 42, 44-55 (1993)). None of the CD40⁻ T cell recipients were diabetic after 45 days (FIG. 2A). Histology of the pancreata confirmed that the islets of CD40⁺ recipients were heavily infiltrated and insulin production diminished by 15 days (FIG. 2B), while pancreata from CD4⁺CD40⁻ control recipients demonstrated no T cell infiltration (FIG. 2C). Injected T cells were determined to be CD8⁻. Furthermore, while CD8⁺ TCR transgenic NOD mice develop diabetes, that process is independent of CD40-CD154 interactions (Amrani, A. et al., (2002)).

CD4⁺CD40⁺ T cells increase in diabetes-prone NOD mice. Because primary CD4^{lo}CD40+ T cells are diabetogenic, we determined the levels of CD4⁺CD40⁺ T cells as auto-immune-prone NOD mice age. We compared levels of these cells in NOD to the diabetes resistant NOR strain and the non-auto-immune BALB/c strain. NOR serves as an important control because these animals contain the same unique MHC configuration, IA^{g7} but are congenic at other loci and do not develop diabetes (Serreze, D. V. et al., J. Exp. Med. 180, 1553-8 (1994)).

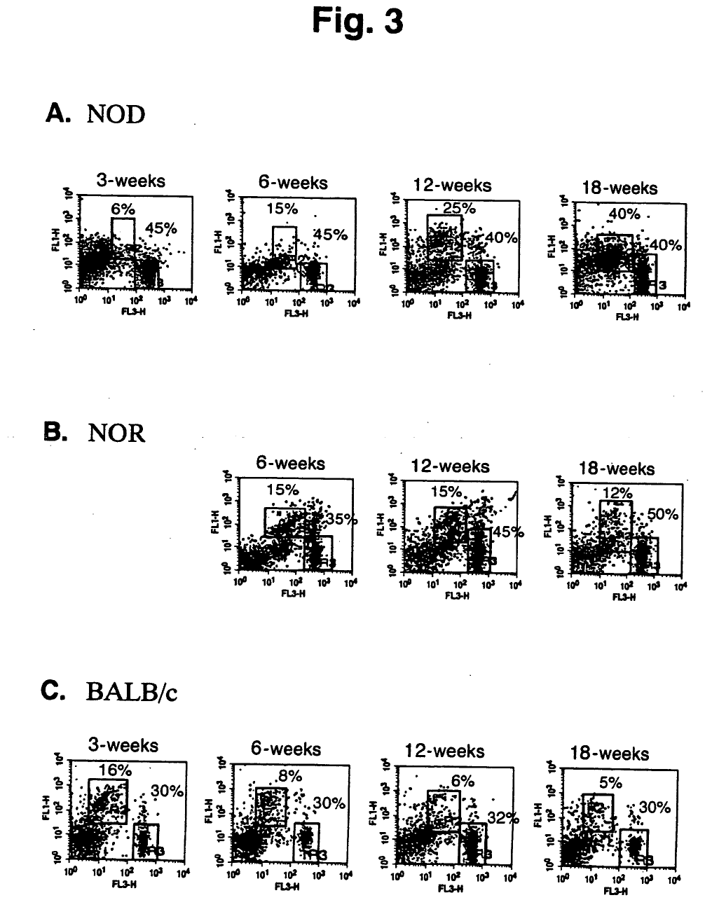

Cells infiltrate the pancreata of NOD mice at 3-weeks of age with progressive insulitis at 12-weeks and diabetes onset typically by 16-20 weeks (Luhder, F. et al., (1998); Baker, F. J. et al., Proc. Natl. Acad. Sci. USA 99, 9374-9 (2002); Szanya, V. et al., J. Immunol. 169, 2461-5 (2002)). In 3-week old NOD females, there were low levels (6%) of CD4^{lo}CD40⁺ T cells (FIG. 3A). The percentage of CD4^{lo}CD40⁺ T cells doubled at 6-weeks of age and by 12-weeks the number increased to 25% of the T cell compartment (FIG. 3A). By 18-weeks the percentage was 40% of the T cell compartment in mice which had not yet become diabetic (FIG. 3A). Over this developmental period, percentages of CD4^{hi}CD40⁻ T cells decreased (FIG. 3A). In diabetic NOD mice, greater than 50% of the CD4⁺ T cell population is CD40+. In the NOR strain, 15% of the T cell population at 6-weeks of age, was CD4^{lo}CD40⁺ and remained consistently at 15% as NOR mice developed (FIG. 3B). Percentages of the CD4^{hi}CD40⁺ T cell population increased through development. Interestingly, CD4^{lo}CD40⁺ T cells in non-auto-immune prone BALB/c mice were highest at 3-weeks of age, 16%, decreasing to 5% as BALB/c mice matured through 18 weeks (FIG. 3C). Reportedly, BALB/c mice contain super-antigens (sAg) that delete specific TCR bearing T cells (Goldman, A. et al., Medicina 55, 45-7 (1995); Maillard, I. et al., Eur. J. Immunol. 26, 1000-6 (1996)). Possibly then, sAg induced depletion accounts for the reduction of CD4⁺CD40⁺ T cells as BALB/c mice age. However the CD4^{hi}CD40⁻ population remained constant.

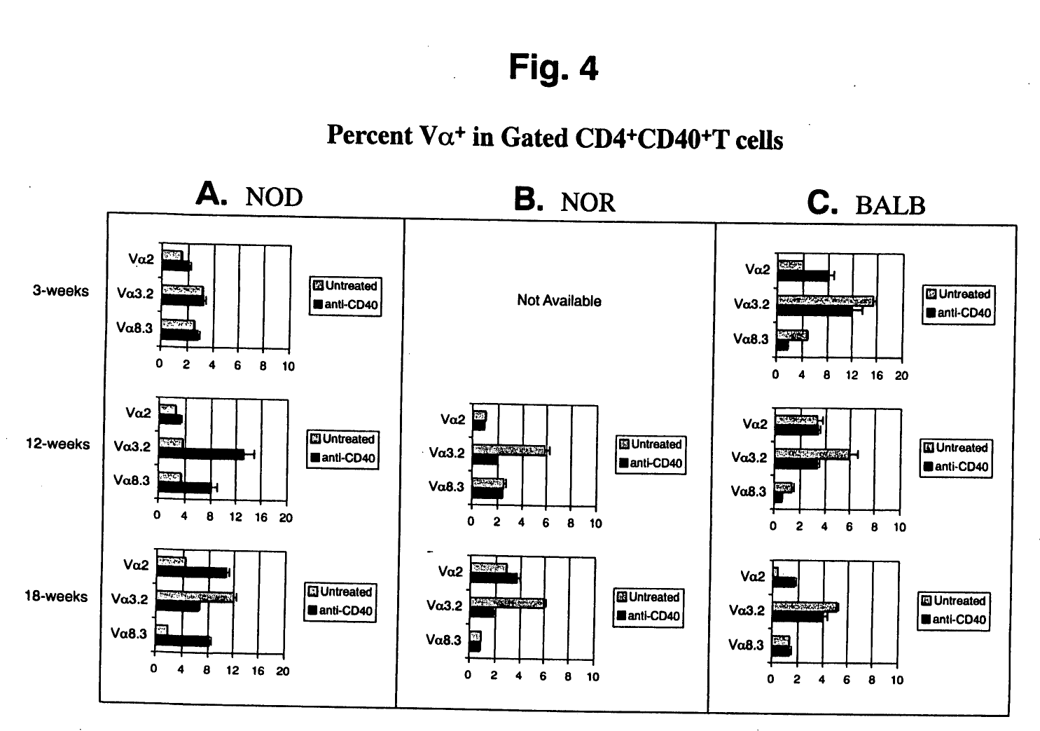

Vα expansions of CD40⁺CD4⁺ T cells in auto-immune NOD mice. Studies of T cells in diabetes have focused largely on diabetogenic T cell clones such as BDC2.5 (Haskins, K. & Wegmann, D. (1996); Luhder, F. et al., (1998)). Even though the BDC2.5 T cell clone is highly diabetogenic, it was recently shown using an anti-idiotype antibody that the BDC2.5 TCR, Vβ4/Vα1, occurs at extremely low levels in the NOD mouse (Kanagawa, 0. et al., J. Immunol. 168, 6159-64 (2002)). Thus another approach is required to study primary T cells as disease culprits. Immediately ex vivo (untreated) CD4⁺CD40⁺ T cells from NOD mice at 3-weeks of age showed that few detectable Vα⁺ T cells were present, with each Vα⁺ population constituting less than 3.5% of the CD4⁺CD40⁺ subset (FIG. 4A). At 12-weeks of age, immediately ex vivo cells showed no significant change in percentages of the Vα⁺ T cells. However, in vitro CD40 cross-linking of T cells induced substantial increases, almost 4-fold, in Vα3.2⁺ and Vα8.3⁺ T cells. These changes were not due to induced selective survival as reported earlier (Vaitaitis, G. M. et al., (2003)) and changes occurred after only 18 hrs. Furthermore, CD40 cross-linking did not induce T cells into cell-cycle as determined by CFSE staining (data not shown). In NOD mice at 18-weeks of age, but not diabetic, there were expansions, when compared to Vα⁺ levels of 3-week old animals, of Vα2⁺ T cells but substantial increases of Vα3.2⁺ T cells in immediately ex vivo cells. Thus these particular T cells expanded in vivo as NOD mice age. In vitro CD40 cross-linking of CD4⁺CD40⁺ T cells induced further changes in Vα expression resulting in increased percentages of Vα2⁺ and Vα8.3⁺ expressing T cells. The CD40⁺ T cells were not propelled into cell cycle as determined by CFSE labeling (data not shown). In older NOD mice, CD40 cross-linking induced reductions in the percentage of Vα3.2⁺ T cells (FIG. 4A). Importantly, T cells were not induced into cell death (data not shown). NOR mice contain the unique MHC-class II component, I-A^{g7} suggesting a similar T cell selective environment to the NOD, however congenic differences at the gene loci that render these animals resistant to development of diabetes (Serreze, D. V. et al., (1994)) may affect T cell development. As demonstrated in FIG. 3, CD4^{lo}CD40⁺ T cells are increased in NOR mice, but only achieve 15% of the total T cell population. At 12-weeks and at 18-weeks of age, NOR animals had higher in vivo levels of Vα3.2⁺ T cells, relative to the other Vα⁺ cells examined. The levels were still lower than in NOD (note scales). Unlike in NOD animals CD40 cross-linking of T cells in both cases induced reductions of Vα3.2⁺ T cells. Again, this was not due to induced cell death (data not shown). The only explanation is that CD40 induced altered expression of Vα consistent with our recent report (Vaitaitis, G. M. et al., (2003)).

In 3-week old BALB/c animals there were low percentages, less than 4%, of the examined Vα⁺ T cells within immediately ex vivo CD4⁺CD40⁻ cells (FIG. 4). However, in vitro CD40 cross-linking induced substantial increases in Vα3.2⁺ and Vα8.3⁺ T cells. At 12-weeks of age within immediately ex vivo T cells there were higher percentages of Vα2⁺ and Vα3.2⁺ T cells compared to levels at 3-weeks of age (FIG. 4). In vitro CD40 engagement had no significant effect on the percentages of Vα2⁺ T cells, but CD40 engagement induced a significant reduction in Vα3.2⁺ T cells. As before, this reduction was not due to induced cell death (data not shown). In older BALB/c animals immediately ex vivo CD4^{lo}CD40⁺ T cells showed higher percentages of Vα3.2⁺ T cells relative to the other examined Vα⁺ T cells. As in 12-week old mice, CD40 engagement induced decreases in levels of Vα3.2⁺ T cells.

Vα3.2⁺ CD4⁺CD40⁺ T cells are increased in pancreas of pre-diabetic and recently diabetic NOD mice. If a specific Vα⁺ T cells were involved in progression of diabetes that cell should be present in pancreata. We also determined Vα⁺ expansions from CD4^{lo}CD40⁺ NOD.scid recipients after onset of diabetes.

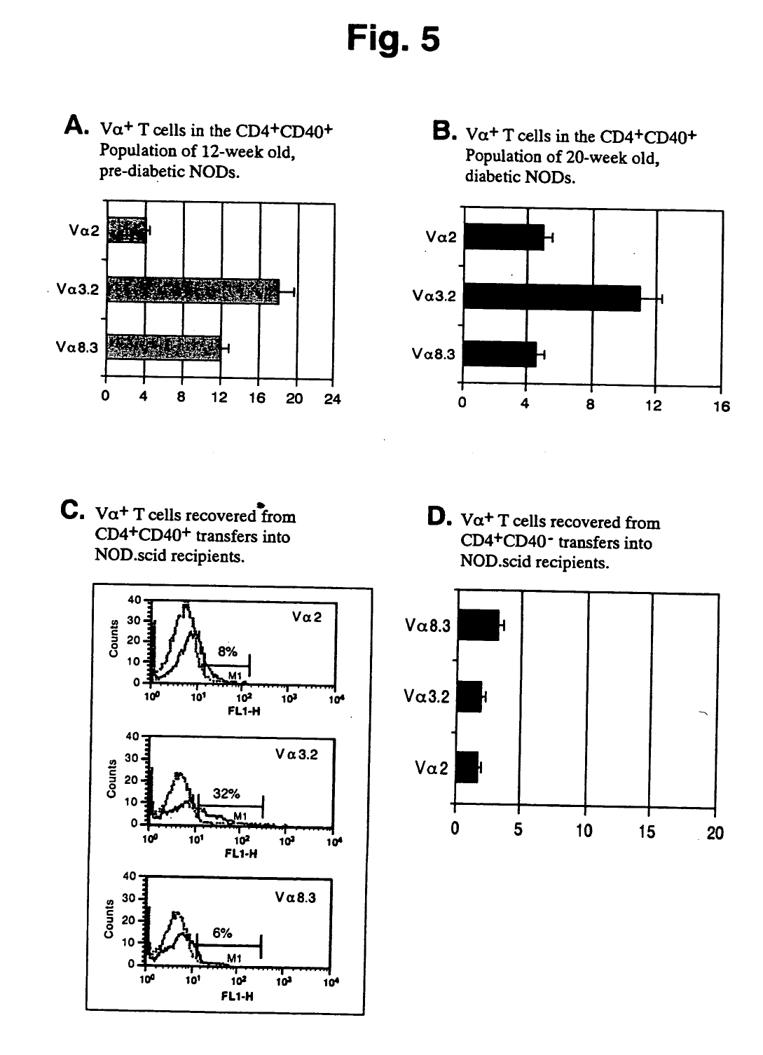

Pancreata from 12-week old, NOD mice showed higher percentages of Vα3.2⁺ and Vα8.3⁺ T cells within the CD4^{lo}CD40⁺, auto-aggressive T cell population (FIG. 5A). Pancreata from newly diagnosed diabetic NOD mice demonstrated an increased percentage of Vα3.2⁺ T cells (FIG. 5B). After diabetes onset within the CD4⁺CD40⁺ recipients, analysis revealed expansions of Vα3.2⁺ cells, comprising 32% within the CD4⁺CD40⁺ T cell population (FIG. 5C). T cells from CD4⁺CD40⁻ recipients demonstrated levels of the Vα⁺ T cells at <4% (FIG. 5D). These data cumulatively suggest that expansions of specific Vα⁺ T cells are associated with, if not directly responsible for, diabetes.

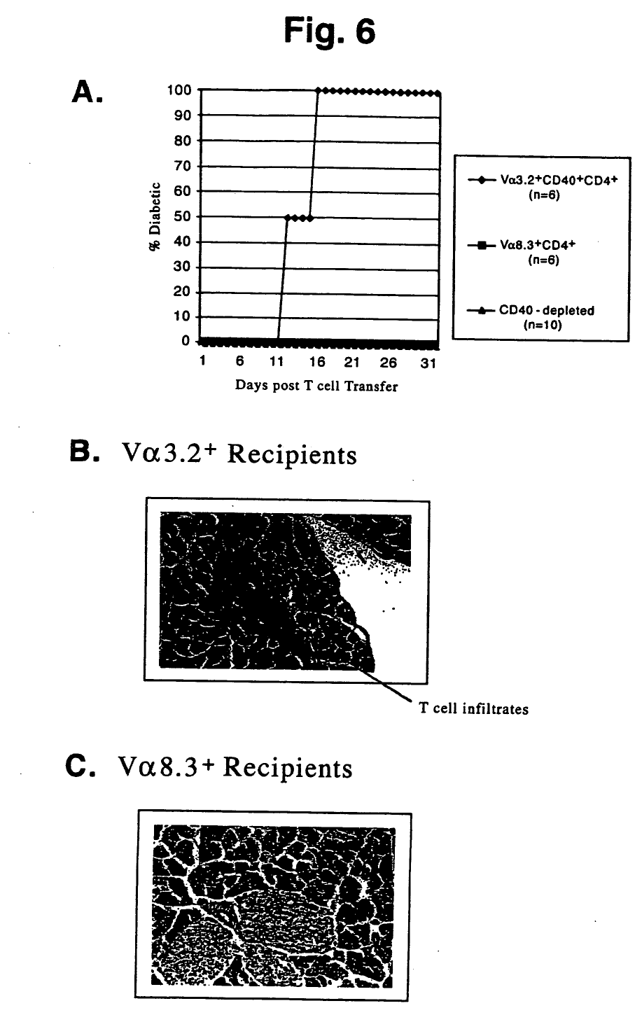

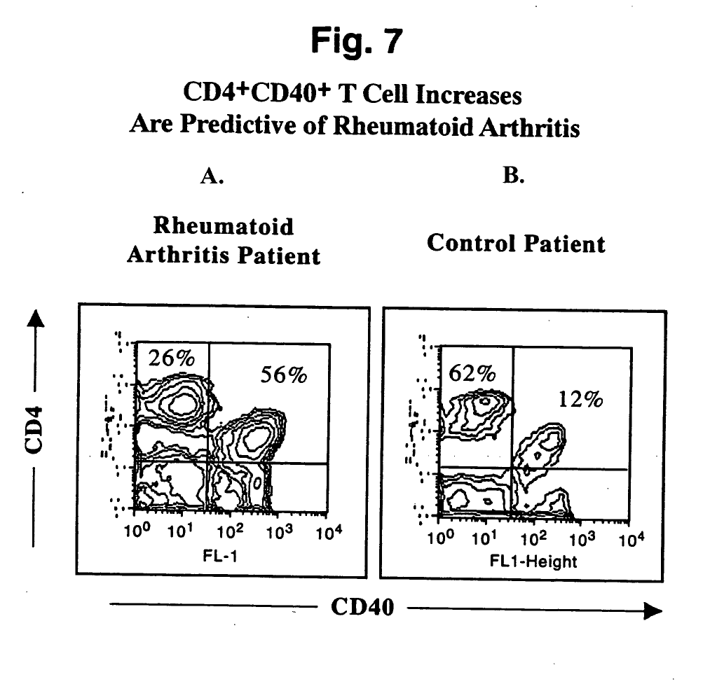

Vα3.2⁺ T cells are highly diabetogenic while Vα8.3⁺ T cells are not. We determined the pathogenicity of Vα3.2⁺ or Vα8.3⁺ T cells through adoptive transfers into NOD.scid recipients. Vα3.2⁺ recipients became diabetic with the same kinetics as recipients of purified CD40⁺ T cells (FIG. 6). That is, 3 of the 6 recipients were diabetic 10-days after injection with 3 more becoming diabetic at 12 days after injection (FIG. 6). These T cells were determined to be CD8⁻. After 45 days, none of the Vα8.3⁺ recipients (6 of 6) and none of the CD4⁺CD40⁻ T cell recipients (10 of 10) became diabetic (FIG. 6). While it is not possible to call these primary T cells a true clonal expansion since they may express different VP molecules, the kinetics of disease transfer is similar to that of established diabetogenic T cell clones (Haskins, K. & Wegmann, D. (1996)). Histology of pancreata from Vα3.2⁺ and Vα8.3⁺ T cell recipients confirmed that Vα3.2⁺ T cells migrate to the pancreas, infiltrate islets and diminish insulin production (FIG. 7A). Conversely, Vα8.3⁺ T cells, examined at 15 days, do not infiltrate the pancreas (FIG. 7B). This study now demonstrates that appropriate isolation of auto-aggressive T cells can be accomplished prior to the onset of diabetes. This also is the first report of primary T cells able to induce diabetes as rapidly as diabetogenic T cell clones.

Discussion

The finding of CD40 involvement in auto-immunity continues to expand. CD40 interactions with its ligand, CD154, have been demonstrated as instrumental in rheumatoid arthritis (Durie, F. H. et al., (1993)), SLE (Wang, X. et al., (2002)), chronic colitis (De Jong, Y. et al., (2000)), atherosclerosis (Lutgens, E. et al., (1999)), scleroderma (Valentini, G. et al., J. Autoimmun. 15, 61-6 (2000)) and several reports demonstrate a definitive role for CD40 signals in T1D. Blocking CD40-CD154 interactions prevents rapid rejection of transplanted islets (Molano, R. et al., Diabetes 50, 270-276 (2001); Kover, K. et al., Diabetes 49, 1666-1670 (2000)). Relative to disease onset, blocking CD40-CD154 interactions early (3-weeks) during NOD development but not later (9-weeks) prevents diabetes (Balasa, B. et al., (1997)). That particular study suggests that an important cell developmental event occurs after 3-weeks but before 9-weeks of age in the auto-immune NOD model. This prompted the current course of study for the newly described CD4^{lo}CD40⁺, auto-aggressive T cell population.

CD40 is expressed on a wide variety of tissues including epithelium (van Den Berg, T. K. et al., Immunol. 88, 294-300 (1996)), endothelium (Kotowicz, K. et al., Immunol. 100, 441-8 (2000)), neural tissue (Suo, Z. et al., J. Neurochem. 80, 655-66 (2002)) and cells of leukocytic origin (Banchereau, J. et al., Ann. Rev. Immunol. 12, 881-920 (1994)). We previously demonstrated that CD40 is expressed on several highly diabetogenic T cell clones; furthermore, we demonstrated that a sub-population of T cells characterized as CD4^{lo}CD40⁺ occur in high numbers in diabetic NOD mice, and successfully transfer diabetes to NOD.scid recipients (Wagner, D. H., Jr. et al., (2002)). In a recent report, we demonstrated that CD40 signals induce transcription, translation, and nuclear translocation of the RAG1 and RAG2 recombinase proteins in peripheral T cells (Vaitaitis, G. M. et al., (2003)). RAGs are responsible for V, D, J recombination of the TCR and subsequent antigen diversity of the T cell repertoire.

Therefore reactivation of RAGs could result in altered TCR expression in peripheral T cells thus escaping thymic negative selection. It is important, however, to recognize CD40⁺ T cells as a sub-population of the T cell compartment because CD40^{-/-} mice still develop T cells though their adaptive immune response including T cell antigen recall is highly impaired (Borrow, P. et al., J. Exp. Med. 183, 2129-42 (1996); Soong, L. et al., Immunity 4, 263-73 (1996)). There are reports of CD40-expressing CD8⁺ T cells (Bourgeois, C. et al., Science 297, 2060-3 (2002)). Relative to diabetes it was demonstrated using a well-described CD8⁺ TCR-Tg model, that CD40-CD154 interactions are not involved in CD8⁺ T cell mediated diabetes onset (Amrani, A. et al., (2002)).

Until now it has been difficult to assess primary T cells as disease culprits in diabetes. It has been reported that transfer of diabetes using primary T cells required that the T cells be isolated from diabetic NOD mice (Christianson, S. W. et al., (1993)). However, in that system extraordinarily large numbers of T cells and both CD4⁺ and CD8⁺ T cells were required to induce diabetes. Recently, it was demonstrated that transfer of highly purified primary CD8⁺ T cells from diabetic NOD mice to NOD.scid recipients did not induce diabetes until primary CD4⁺ T cells were transferred (Lejon, K. & Fathman, C. G., (1999)). There likely are multiple ways of inducing diabetes involving several different cellular mechanisms. Complicating this picture, there are highly successful diabetogenic CD8⁺ T cell clones and subsequent TCR-Tg animals, which do not appear to require CD4⁺ help (Anderson, B. et al., Proc. Natl. Acad. Sci. USA 96, 9311-6 (1999); Serra, P. et al., Proc. Natl. Acad. Sci. USA 13, 13 (2002)).

The involvement of CD4⁺ T cells in T1D has focused largely on diabetogenic T cell clones e.g., BDC2.5 and the corresponding BDC2.5 TCR-Tg animal (Katz, J. D. (1993)). Although BDC2.5 rapidly transfers disease, recently it was reported that its clonally defined TCR, Vβ4/Vα1 is grossly under-represented within NOD mice including the BDC2.5 TCR-Tg animal (Kanagawa, 0. et al., (2002)). Theoretically, clonal expansions would occur due to availability of self-antigens. However, it is possible that changes within the TCR, such that it is no longer detectable by anti-idiotype antibody occurs, but these T cells remain diabetogenic. Another study demonstrated that within the BDC2.5TCR-Tg animal there is substantial drift within Vα usage but animals become diabetic nevertheless (Luhder, F. et al., (1998)). The current report demonstrates that auto-aggressive T cells expand as NOD mice age, likely by an antigen-driven response. Additionally, there are CD40-driven expansions of Vα3.2⁺ cells within NOD T cells. Interestingly, in the NOR control there were early expansions of Vα3.2⁺ T cells but only relative to the other Vα⁺ T cells examined. The levels of Vα3.2⁺ T cells were substantially lower. Because the CD4^{lo}CD40⁺ T cell population does not expand in NOR, the numbers of Vα3.2⁺ T cells potentially do not reach a critical mass to induce disease. Nevertheless, these data suggest that changes in TCR relative to Vα expression are intrinsic to diabetogenesis.

There are two possible scenarios to explain the Vα increases within the periphery, proliferation or alteration in Vα expression. We have determined that CD40 signals do not promote T cells into cell cycle. In addition, CD40 signals promote T cell survival and not selective cell death. We have shown that CD40 signals auto-aggressive T cells to increase RAG1 and RAG2 expression, and importantly, CD40 signals induce translocation of the RAG proteins to the nucleus (Vaitaitis, G. M. et al., (2003)). Therefore the most likely explanation is that CD40 signals induce altered Vα expression, explaining the expansion of Vα3.2⁺ and Vα8.3⁺ T cells. The clonal nature of these cells is indeterminate because the VP repertoire of these cells is as yet unknown. Vα expression may define a subset of T cells that can be further qualified relative to Vβ expression. It has been demonstrated that diabetogenic T cell clones become heterogeneous with respect to antigen specificity (Candeias, S. et al., (1991)) suggesting that several β cell antigens are involved in the diabetogenic process. Therefore the Vα3.2⁺ T cells may express several different Vβ molecules but nonetheless rapidly induce diabetes.

### Example 2

Diagnostic Tests for Auto-Immune Diseases: Type 1 Diabetes

A diagnostic test for type 1 diabetes comprising a blood test determining the levels of CD4⁺CD40⁺ T cells is envisioned. For this diagnostic test, a blood sample or samples comprising T cells is taken from a predetermined subject. Similarly, a blood sample or samples comprising T cells is taken from one or more subjects not having, or prone to develop, type 1 diabetes. The blood sample from the non-prone subject(s) (the control sample or population) establishes the baseline level (control level) of CD4⁺CD40⁺ T cells in the control population.

The cell-containing samples from both populations are treated with a fluorescent anti-CD4 antibody in combination with a fluorescent anti-CD40 antibody and the sample cells are assayed for expression of CD4 and CD40 by flowcytometry using methods known in the art. Levels of CD4⁺CD40⁺ cells in the control sample and the subject sample are determined. Exaggerated levels of CD4⁺CD40⁺ cells are levels higher than those in the control population. Exaggerated levels of CD4⁺CD40⁺ cells indicate a propensity to develop type 1 diabetes.

### Example 3

Diagnostic and Predictive Tests for Emphysema

Emphysema is a chronic obstructive pulmonary disease (COPD) that results in destruction of alveoli of the lungs. The disease is both life altering and life threatening. While most suffers of emphysema are or have been chronic smokers, all smokers do not contract emphysema. This is consistent with auto-immune disease.

Smokers are exposed to tobacco smoke antigens, but not every individual develops emphysema. This invention will specifically test a person's susceptibility to develop COPD by tobacco smoke exposure. Blood will be drawn from an individual and examined for CD4⁺CD40⁺ T cells, the hallmark of disease potential. Lymphocytes will be isolated by standard means, and exposed to tobacco smoke antigens. Simple tests of response including proliferation and T cell cytokine production will be tested using flow cytometry. Cells will be stained directly for expression of CD40 and CD4, then labeled to determine proliferation and stained intra-cellularly for cytokine production. This invention will encompass an approximately 4-5 day test period, at which time positive or negative results can be reported to the requesting physician.

### Example 4

CD4⁺CD40⁺ T cell increases are predictive of rheumatoid arthritis. Peripheral blood, 10 ml, was drawn by phlebotomy from clinically identified rheumatoid arthritis (RA) patients. Blood was mixed with phosphate buffered saline (PBS) 1:1 then layered on Ficoll and centrifuged to isolate lymphocytes. Lymphocytes were collected, washed with PBS and directly stained with Cy-chrome conjugated anti-CD4 and FITC-conjugated anti-CD40. Stained T cells were analyzed using a FACScalibur Flow Cytometer. Levels of T cells were compared from RA patients and control patients. As in type 1 diabetes, CD4⁺CD40⁺ T cell levels are greatly exaggerated, 56% versus 12%, in RA compared to controls. Thus CD4⁺CD40⁺ T cell increases are predictive of rheumatoid arthritis. Results are shown in FIGS. 7A and 7B.

### Example 5

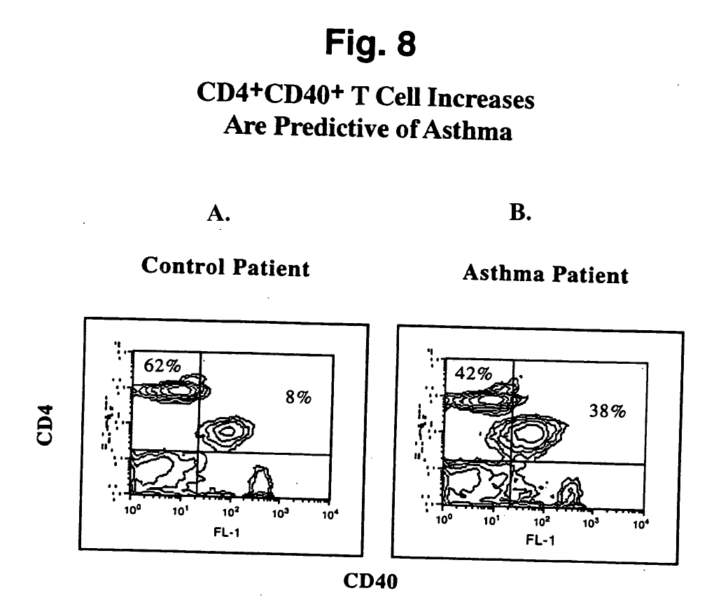

CD4⁺CD40⁺ T cell increases are predictive of asthma. Peripheral blood, 10 ml, was drawn by phlebotomy from clinically identified Asthma patients. Blood was mixed with phosphate buffered saline (PBS) 1:1 then layered on Ficoll and centrifuged to isolate lymphocytes. Lymphocytes were collected, washed with PBS and directly stained with Cy-chrome conjugated anti-CD4 and FITC-conjugated anti-CD40. Stained T cells were analyzed using a FACScalibur Flow Cytometer. Levels of T cells were compared from Asthma patients and control patients. As in type 1 diabetes, CD4⁺CD40⁺ T cell levels are greatly exaggerated, 38% versus 8%, in RA compared to controls. Thus CD4⁺CD40⁺ T cell increases are predictive of asthma. Results are shown in FIGS. 8A and 8B.

### Example 6

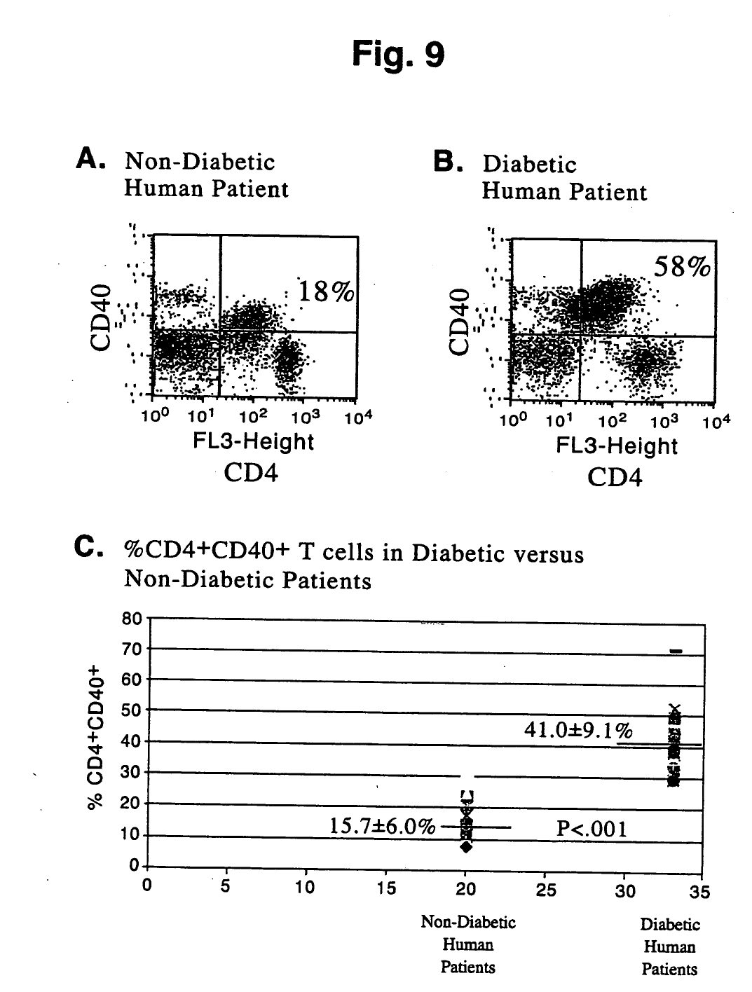

CD40⁺CD4⁺ T cells are predictive for Human type 1 diabetes. Blood was drawn from 25 clinically diagnosed type 1 diabetic patients and from 20 non-diabetic controls. Whole blood was diluted with PBS, suspended on Hypaque-Ficoll, centrifuged for 10 min at 5000 RPM. Leukocytes were isolated and stained with directly conjugated anti-CD3, anti-CD4 and anti-CD40. Cells were assayed through a FACScalibur flow cytometer. Cells were gated on CD3 (T cell marker) and analyzed for CD4 and CD40 levels. Controls (A) and Diabetics (B) are represented. Total percent of CD4⁺CD40⁺/CD4⁺CD40⁺+CD4⁺CD40⁻ are represented (C). This measurement is predictive of diabetes. Results are presented in FIGS. 9A-C.

### Example 7

Materials and Methods

Human T cells: Patients were recruited from the Barbara Davis Childhood Diabetes Center clinic at the University of Colorado Denver and Health Sciences Center. T1D patients included recent onset, within 6-weeks of examination, and long term, up to 20 years after initial diagnosis. The age range of patients was from 19 to 64 years of age, with clustering in 20s and 30s. T1D and T2D were diagnosed by the American Diabetes Association criteria including failed glucose tolerance test and confirmed by anti-islet, auto-antibody positive tests in T1D and no auto-antibody detection in the case of T2D patients. Controls were age and gender matched, and have not clinically presented with any inflammation related complications. Peripheral blood, 10 ml, was collected, then diluted 1:1 with PBS and passed through Ficol-Hypaque. Study includes 25 diagnosed T1D and 25 control patients.

Staining: Human lymphocytes, 1×10⁶, were stained with directly conjugated antibodies including: anti-CD40 (FITC, clone 5C3); anti-CD4 (PE-Cy5, clone RPA-T4); anti-CD3 (PE, clone UCHT1); anti-CD8 (PE-Cy5, clone RPA-T8); and isotype controls all from e-bioscience, San Diego, Calif.

Antigen Stimulation of T cells: Peripheral blood lymphocytes were isolated and passed through nylon wool columns to purify T cells (Wagner, D. H., Jr., Stout, R. D. & Suttles, J. (1994) European Journal of Immunology 24, 3148-3154). Cells eluted from columns were 93% CD3⁺. Antigen presenting cells (APC) and T cells were segregated as described (Folzenlogen, D., Hofer, M. F., Leung, D. Y., Freed, J. H. & Newell, M. K. (1997) Clin Immunol Immunopathol 83, 199-204). Purified APCs, 1×10⁵, were incubated for 8 hrs with 10 μM pre-pro-insulin, amino acid sequence 73-90 including (A G S L Q P L A L E G S L Q K R G (SEQ ID NO: 22)); GAD 271-285, including (P R L I A F T S E H S H F S L (SEQ ID NO: 23)); or GAD 556-570 including (F F R M V I S N P A A T H Q D (SEQ ID NO: 24)) peptides or with 10 picked human islets per well. Foreign antigen, tetanus toxoid at 5 μg/ml was used. PHA-M at 1 μg/ml was included as a positive control. Purified T cells from both T1D and normal controls were rested overnight in RPMI, 5% FCS media, then 1×10⁵ were labeled with 5 μM CFSE for 30 in the dark, washed with PBS-5% FCS and incubated with APC alone or APC+antigens in 0.1 ml RPMI/5% FCS for 48, 72 or 96 hr. After stimulation, T cells (CFSE⁺) were cell surface stained with anti-CD4 (CyChrome 5) and anti-CD40 (PE). Cell proliferation was assayed as loss of CFSE fluorescence measured in FL-1 (Mannering, S. I., Morris, J. S., Jensen, K. P., Purcell, A. W., Honeyman, M. C., van Endert, P. M. & Harrison, L. C. (2003) J Immunol Methods 283, 173-83).

Statistical Analysis: Statistical analysis was performed by Student's T test difference of means on all treated samples. Within the examples, appropriate p values are included.

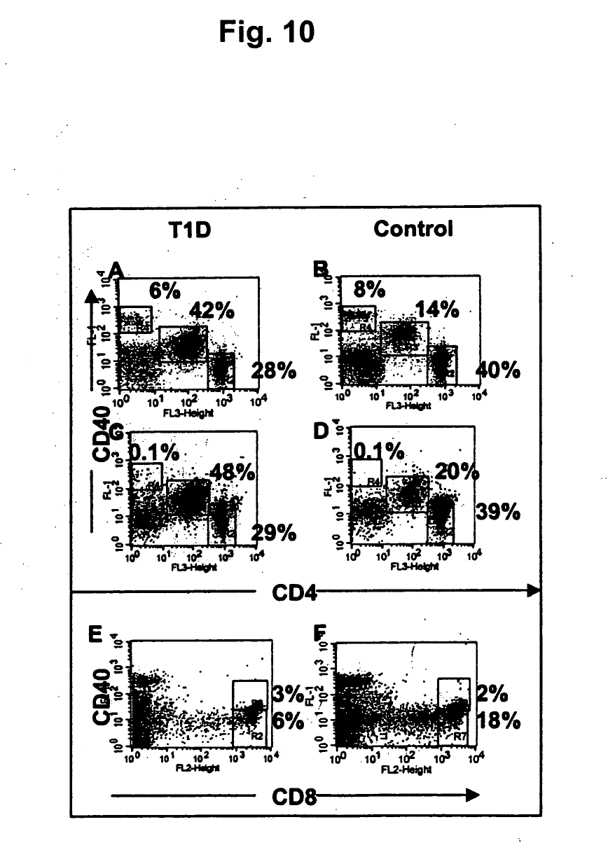

CD4⁺CD40⁺ T Cells are Expanded in the Peripheral Dlood of T1D Patients. We previously characterized a unique Th subset, CD4⁺CD40⁺, in the NOD mouse diabetes model as harboring highly pathogenic T cells. Importantly, these T cells but not CD4⁺CD40⁻ T cells, expand concurrently with progressive insulitis, and when purified, rapidly transfer severe insulitis and hyperglycemia to immune-compromised recipient mice. We explored the possibility that CD4⁺CD40⁺ T cells are expanded in peripheral blood of T1D patients. We compared levels of CD4⁺CD40⁺ T cells in PBL of T1D and control, non-autoimmune patients in a blinded study. Blood samples from human patients were collected at the Barbara Davis Childhood Diabetes Center, Denver, Colo. and analyzed. As demonstrated in the NOD mouse diabetes model, peripheral blood lymphocytes (PBL) of T1D patients exhibit a substantial increase in the Th subset, CD4⁺CD40⁺ T cells (45% of total PBL) (FIG. 10A) compared to controls (15% of PBL) (FIG. 10B). Interestingly, the levels in T1D patients are equivalent to that of diabetic NOD mice and the levels in control patients match those of naïve, non-autoimmune mouse strains. There are reports of CD8⁺ pathogenic T cells contributing to diabetes in the NOD model and CD8⁺CD40⁺ T cells have been reported. However no discernable differences in CD8⁺CD40⁺ T cell levels between T1D and control patients were detected (FIGS. 10C and D) and remained consistent at 5% of PBL. This suggests that CD8⁺CD40⁺ T cells may be less prominent in human diabetes.

We gated on CD3⁺ to confirm that CD4⁺CD40⁺ cells are a T cell subset. CD4^{lo}CD40⁺ cells clearly fall within the CD3⁺ subset, thus restricting them to a T cell phenotype (FIGS. 10E and F). A sub-population of CD40⁺ cells, likely representing B cells, is gated out. There were no discernable differences in levels, 10% in both T1D and control patients, of CD4-CD40⁺ B cells.

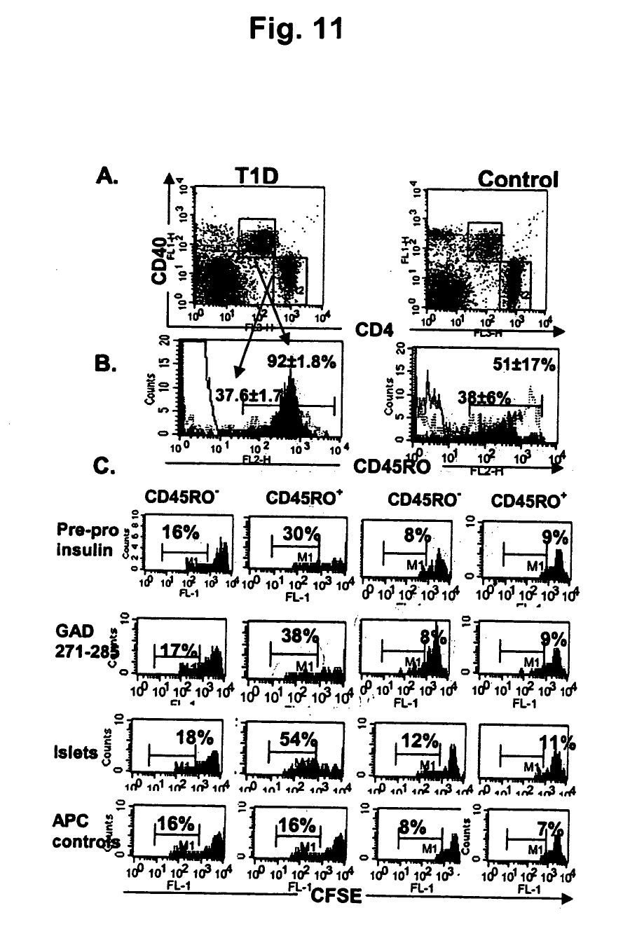

CD4⁺CD40⁺ T cells are Antigen Responsive and able to Attain Effector Status. CD4⁺CD40⁺ and CD4⁺CD40⁻ T cells from T1D and control patients were exposed to the tetanus toxoid. In antigen recall assays, CD4⁺CD40⁺ and CD4⁺CD40⁻ T cells mount a proliferative response when exposed to antigen presenting cells (APC) and tetanus toxoid (FIGS. 11A and B). Interestingly, CD4⁺CD40⁺ T cells from T1D consistently were more responsive than those from non-autoimmune patients suggesting that CD4⁺CD40⁺ T cells from T1D may be more easily primed for effector response. To determine if CD4⁺CD40⁺ T cells have auto-aggressor potential we explored their response to human islets, pre-pro-insulin, and two GAD peptides. Recent reports suggest that pre-pro-insulin (PPI) and glutamic acid decarboxylase (GAD) are diabetes associated self-antigens. We show here that T1D CD4⁺CD40⁺ T cells have marked proliferative response to whole human islets while the identical population from control patients does not respond (FIG. 11C). Because T cell proliferation is dependent upon TCR recognition of specific presented antigens, a portion of CD4⁺CD40⁺ T cells from T1D patients must possess islet-antigen restricted, auto-aggressive TCR molecules. This suggests that during T1D, these T cells can initiate the auto-aggressive attack leading to β cell destruction. These T cells further were responsive to the PPI and GAD peptides while T cells from non-autoimmune patients were completely non-responsive (FIG. 11C). This suggests that the T1D CD4⁺CD40⁺ T cell population is comprised of different auto-aggressor TCR bearing T cells that therefore make up a pathogenic subset of T cells.

CD4⁺CD40⁻ T cells from both T1D and control patients statistically were not responsive to the self-antigens tested suggesting that these T cells do not harbor diabetogenic T cells (FIG. 11C). This finding is consistent with the observation that CD4⁺CD40⁻ T cells in the NOD diabetes model are non pathogenic. We confirmed that these T cells were not anergic as CD4⁺CD40⁻ T cells from both T1D and controls were significantly responsive to PHA, a nonspecific T cell activator.

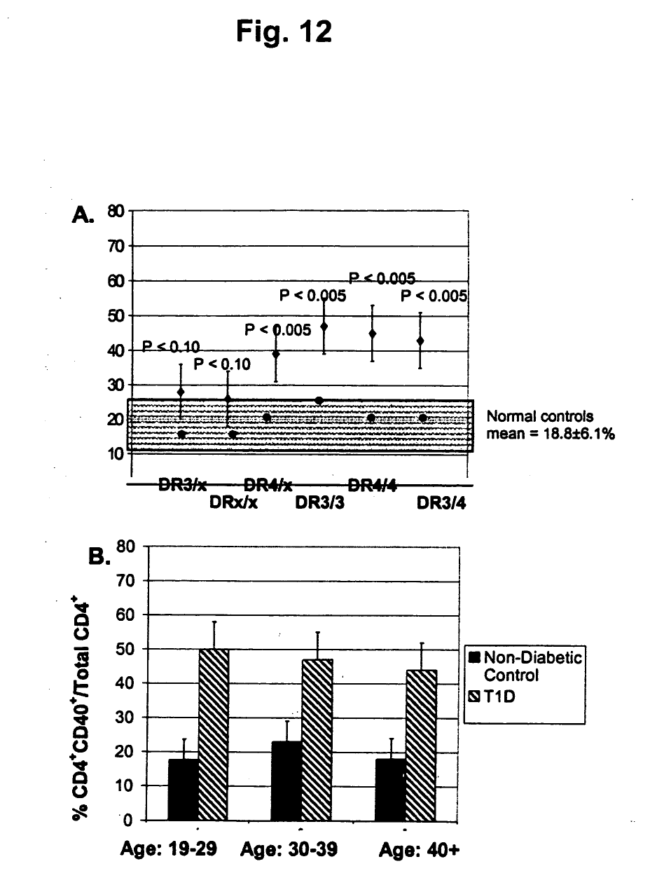

Expanded Levels of CD4⁺CD40⁺ T cells Occur Independently of the Expression of the Diabetes-Associated HLA-DR and Persistence of Hyperglycemia. HLA expression has been tightly associated with autoimmunity and the highest risk group for T1D is reportedly HLA-DR3/4 DQ8. Numerous studies correlate HLA-DR3 and HLA-DR4 expression with T1D. We therefore determined if there is a correlation between percent of CD4⁺CD40⁺ T cells and HLA-DR expression. We discovered that the highest percentages of CD4⁺CD40⁺ T cells occurred in patients that contained either DR3 or DR4 at both alleles, i.e., DR3/DR3, DR4/DR4 or DR3/DR4 (FIG. 12A). The difference in CD4⁺CD40⁺ T cell levels within these HLA-DRs between T1D and control patients was highly significant (p<0.001 by Student's T test difference of means). In T1D patients, when HLA-DR4 occurred at only one allele (DR4/x), CD4⁺CD40⁺ T cell levels remained significantly above control levels (p<0.005). When one allele was DR3 and the other was any other than DR4 or DR3 (DR3/x), or both alleles were anything other than DR3 or DR4 (DRx/x), CD4⁺CD40⁺ T cell levels were not significantly higher than controls (FIG. 12A). Non-autoimmune controls exhibited a wide range of DR alleles, including DR4/x, DR4/4 and DR4/3 but levels of CD4⁺CD40⁺ T cells remain contained (FIG. 12A). Levels of CD4⁺CD40⁺ T cells were 23%±2.1 when HLA-DR4/x alleles occurred, and were 21.3%±3.1 when DR4/4 or DR4/3 occurred.

We determined correlation between age of patients and CD4⁺CD40⁺ T cell levels with the concern that expanded CD4⁺CD40⁺ T cells may correspond to persistent hyperglycemia. The age range of patients was from 19 to 64 years, with the majority of patients falling between 20 and 40 years old. The ratio of CD4⁺CD40⁺ T cells in T1D patients compared to age-matched controls was highest at younger ages (FIG. 12B). Although not significantly different between diabetic patient subsets (p>0.25), but significantly above age matched controls, levels of CD4⁺CD40⁺ T cells slightly decrease with increasing age-groups of T1D patients (FIG. 12B). In older patients (40+), T1D was diagnosed more than 20 years previously while disease onset was of recent onset (6 months-5 years) in younger (19-29 year old) patients.

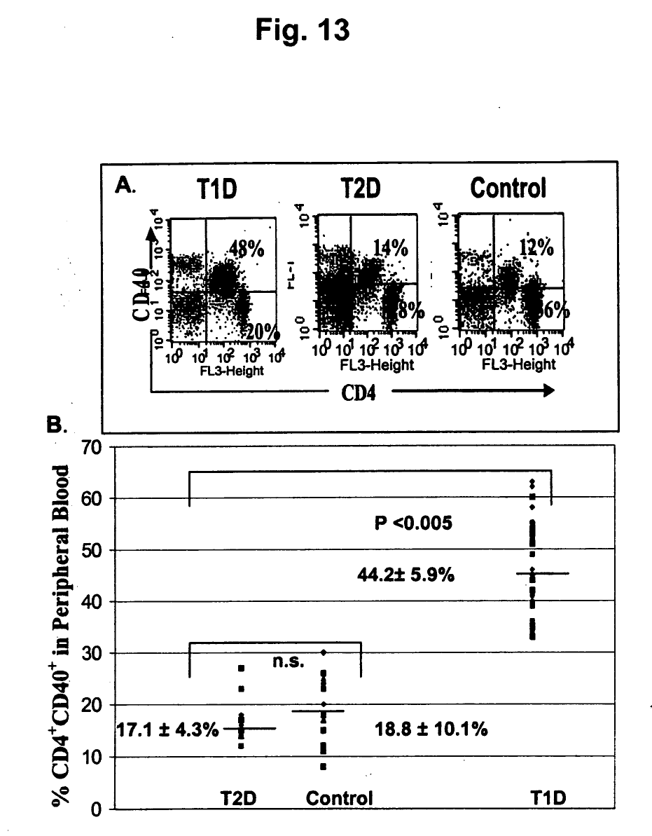

The Expanded CD4⁺CD40⁺ T cell Subset in T1D is Independent of Hyperglycemia. The issue of CD4⁺CD40⁺ T cell expansion and hyperglycemia can be addressed further by comparing type 1 and type 2 diabetic (T2D) patients. While clinically similar relative to hyperglycemia, T1D is an autoimmune disease and T2D is not. T1D is insulin dependent due to the auto-aggressor cell destruction of the islet β cells, while in T2D there is only modest loss of β cells and insulin dependence usually develops only after years of diabetes. When PBL from clinically diagnosed T2D patients are examined, peripheral CD4⁺CD40⁺ T cells remain contained at levels equivalent to controls (FIG. 13A). The mean level of CD4⁺CD40⁺ cells for T1D patients examined is 44.2% of PBL while the level for non-autoimmune patients is 18.8%. The level of CD4⁺CD40⁺ cells for T2D is 15.4%±2.0%, which is not statistically different from controls, but highly significantly different from T1D levels (FIG. 13B).

Diagnostic Tests for Distinguishing Type 1 from Type 2 Diabetes. A diagnostic test for distinguishing type 1 from type 2 diabetes comprising a blood test determining the levels of CD4⁺CD40⁺ T cells is envisioned. For this diagnostic test, a blood sample or samples comprising T cells is taken from a predetermined subject. Similarly, a blood sample or samples comprising T cells is taken from one or more subjects not having, or prone to develop, type 1 diabetes or having type 2 diabetes. The blood sample from the non-prone subject(s) or subjects with type 2 diabetes (the control sample or population) establishes the baseline level (control level) of CD4⁺CD40⁺ T cells in the control population.

The cell-containing samples from both populations are treated with a fluorescent anti-CD4 antibody in combination with a fluorescent anti-CD40 antibody and the sample cells are assayed for expression of CD4 and CD40 by flowcytometry using methods known in the art. Levels of CD4⁺CD40⁺ cells in the control sample and the subject sample are determined. Exaggerated levels of CD4⁺CD40⁺ cells are levels higher than those in the control population. Exaggerated levels of CD4⁺CD40⁺ cells indicate the perdetermined subject has type 1 diabetes.

Discussion

This present invention describes a phenotypically unique human T cell subset, CD4⁺ CD40⁺ T cells, which are potentially auto-aggressor in T1D. Previously, we identified CD4⁺ CD40⁺T cells as a pathogenic effector T cell subset in the NOD T1D model. We showed that in NOD mice, concurrent with progressive insulitis, CD4⁺CD40⁺ T cells continually expand to greater than 50% of the T cell compartment by diabetes onset (Wagner, D. H., Jr., Vaitaitis, G., Sanderson, R., Poulin, M., Dobbs, C. & Haskins, K. (2002) Proc Natl Acad Sci USA 99, 3782-7; Vaitaitis, G. M., Poulin, M., Sanderson, R. J., Haskins, K. J. & Wagner Jr., D. H. (2003) Cutting Edge, J. Immunol. 170, 3455-3459; Waid, D. M., Vaitaitis, G. M. & Wagner, J., D. H. (2004) European Journal of Immunology 34, 1488-1497; Bach, J. F. & Chatenoud, L. (2001) Annu Rev Immunol 19, 131-61). There is no expansion in the non-diabetic NOD congenic NOR strain, or in the Balb/c, non-autoimmune strain. Pathogenicity of this T cell subset was defined by multiple adoptive transfer studies including isolation from diabetic and pre-diabetic NOD mice (Wagner et al. (2002); Vaitaitis et al. (2003); Waid et al. (2004); and Bach et al. (2001)). Importantly CD4⁺CD40⁻ T cells do not transfer insulitis or diabetes (Wagner et al. (2002); and Waid et al. (2004)).

CD40-CD154 interaction as causal in T1D has been strongly suggested (Homann, D., Jahreis, A., Wolfe, T., Hughes, A., Coon, B., van Stipdonk, M. J., Prilliman, K. R., Schoenberger, S. P. & von Herrath, M. G. (2002) Immunity 16, 403-15; Balasa, B., Krahl, T., Patstone, G., Lee, J., Tisch, R., McDevitt, H. O. & Sarvetnick, N. (1997) Journal of Immunology 159, 4620-7; Becher, B., Durell, B., Miga, A., Hickey, W. & Noelle, R. (2001) J. Exp. Med. 193, 967-974; Durie, F. H., Fava, R. A., Foy, T. M., Aruffo, A., Ledbetter, J. A. & Noelle, R. J. (1993) Science 281, 1328-1330). Blocking CD40-CD154 interaction at 3-weeks but not 9-weeks of age in NOD mice prevents T1D (Balasa, B., Krahl, T., Patstone, G., Lee, J., Tisch, R., McDevitt, H. O. & Sarvetnick, N. (1997) Journal of Immunology 159, 4620-7). Also, mice lacking CD154 (CD154^{-/-}), the CD40 ligand, do not develop T1D (Amrani, A., Serra, P., Yamanouchi, J., Han, B., Thiessen, S., Verdaguer, J. & Santamaria, P. (2002) Immunity 16, 719-32). We mechanistically demonstrated that blocking CD40-CD154 interaction prevents expansion of CD4⁺CD40⁺ T cells but allows expansion of a described regulatory CD4⁺CD25⁺CD69⁻ (Berzins, S. P., Venanzi, E. S., Benoist, C. & Mathis, D. (2003) Diabetes 52, 327-34) T cell subset (Waid et al. (2004).

Mirroring the mouse studies, human T1D peripheral CD4⁺CD40⁺ T cells are significantly expanded and while the cells are present in controls, they remain contained. Importantly the expansion of CD4⁺CD40⁺ T cells in T1D is highest in patients expressing either the HLA-DR4 or DR3 alleles, which is strongly associated with T1D. Control, non-autoimmune patients also expressed DR4 or DR3, but the CD4⁺CD40⁺ T cell population remained contained. This suggests that independently of HLA-DR expression, expanded CD4⁺CD40⁺ T cells are indicative of T1D. Furthermore, the expanded percentage of CD4⁺CD40⁺ T cells occurred regardless of age of the patient, and was even slightly higher in younger, recent onset patients.

A recent report described the isolation of insulin responsive, HLA-DR4 restricted T cell clones from pancreatic lymph nodes of T1D patients (Kent, S. C., Chen, Y., Bregoli, L., Clemmings, S. M., Kenyon, N. S., Ricordi, C., Hering, B. J. & Hafler, D. A. (2005) Nature 435, 224-8). While that data are revealing, the current report indicates that auto-aggressor T cells may be detected in peripheral blood and not just in the pancreatic lymph nodes which can only be assayed post mortem. We further demonstrate here the ability of CD4⁺CD40⁺ T cells to achieve effector status and auto-antigen responsiveness that is restricted to the autoimmune T1D population. This suggests that CD4⁺CD40⁺ T cells may attack pancreatic β cells in human patients.

An important discovery in this study is that autoimmune T1D patients have expanded CD4⁺CD40⁺ T cells while non-autoimmune T2D patients do not. Even though hyperglycemic, the levels of CD4⁺CD40⁺ T cells in T2D are statistically the same as in non-autoimmune controls. While the initial manifestation of T1D and T2D hyperglycemia is the same, these two diseases have very different clinical courses and therefore it is important to accurately distinguish between them. This study suggests a clear distinction demonstrated by differences in the percentage of the CD4⁺CD40⁺ T cell subset.

It appears that sustained high levels of CD40⁺ T cells promote the generation of auto-aggressor T cells. We showed in NOD mice that CD40 engagement induces RAG1/RAG2, the recombinase proteins required for altering TCR expression, in peripheral T cells (Vaitaitis et al. (2003)). This process is called TCR revision (Cooper, C. J., Turk, G. L., Sun, M., Farr, A. G. & Fink, P. J. (2004) J Immunol 173, 6532-6; Ali, M., Weinreich, M., Balcaitis, S., Cooper, C. J. & Fink, P. J. (2003) J Immunol 171, 6290-6; Cooper, C. J., Orr, M. T., McMahan, C. J. & Fink, P. J. (2003) J Immunol 171, 226-33). We showed that a specific TCR Vα bearing T cell (Vα3.2⁺) occurs at undetectable levels in very young NOD mice and increases to represent 12% of peripheral CD4⁺CD40⁺ T cells by diabetes onset at 16-20 weeks of age (Waid et al. (2004)). We determined that engaging CD40 on T cells from young mice (no detectable levels of Vα3.2⁺ T cells) induced TCR revision leading to Vα3.2 expression. Importantly these TCR revised T cells proved to be highly auto-aggressive, causing rapid diabetes onset in NOD.scid adoptive transfer models (Waid et al. (2004)). If the mouse model mirrors human disease, high levels of CD4⁺CD40⁺ T cells during T1D could be induced to TCR revision in the periphery that potentially leads to expression of auto-aggressor T cells. Furthermore, it appears that CD4⁺CD40⁺ T cells in T1D are capable of responding to several T1D-associated antigens. This suggests a subset of auto-aggressor T cells may prove highly pathogenic in this disease.

All cited patents, patent applications, publications and other documents cited in this application are herein incorporated by reference in their entirety. The present invention is not to be limited in terms of the particular embodiments described in this application, which are intended as single illustrations of individual aspects of the invention. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims.

What is claimed is:

1. A method of determining whether a subject has type 1 diabetes or type 2 diabetes comprising:
a) obtaining a blood sample from the subject;
b) detecting CD4⁺CD40⁺ T cells in the blood sample;
c) determining whether the CD4⁺CD40⁺ T cells in the blood sample are expanded by comparing to CD4⁺CD40⁺ T cells in a control sample of blood, wherein an expanded level of CD4⁺CD40⁺ T cells indicates that the subject has type 1 diabetes.

2. The method of claim 1, wherein CD4⁺CD40⁺ T cells are detected by PCR.

3. The method of claim 1 further comprising contacting the blood sample from the subject and the control with antibodies to both CD4 and CD40.

4. The method of claim 3, wherein CD4⁺CD40⁺ T cells are detected by flow cytometry.

5. The method of claim 3, wherein the CD4⁺CD40⁺ T cells are detected by an immunoassay.

6. The method of claim 5, wherein the immunoassay is an enzyme-linked immunosorbent assay, an immunoflorescence assay, or a radioimmunoassay.

7. The method of claim 1, wherein the blood sample comprises peripheral blood.

8. The method of claim 1, wherein the blood sample consists essentially of peripheral blood.

9. The method of claim 1, wherein the control sample of blood is from a subject diagnosed with type 2 diabetes.

10. The method of claim 1, wherein the control sample of blood is from a subject that does not have diabetes.

11. The method of claim 1, wherein the subject is a mammal.

12. The method of claim 11, wherein the mammal is a human.

13. A kit for determining whether a subject has type 1 diabetes or type 2 diabetes comprising: a control blood sample, means for obtaining blood sample from the subject, means for detecting CD4⁺CD40⁺ T cells, and a means for determining whether the CD4⁺CD40⁺ T cells in the blood sample from the subject are expanded.


SEQUENCE LISTING (24 sequences)

SEQ ID NO: 1 - DNA, 6545 bp, Homo sapiens
  CDS (113)..(3244)
gagagcagagaacacactttgccttctctttggtattgagtaatatcaaccaaattgcagacatctcaacactttggccaggcagcctgctgagcaaggtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggccggccccgccaacatcttctgtcgctgactcggagagctcagaagcaccggctgagggagctcaagctgcaagtcaaagcctttgctgacaaagaagaaggtggagatgtgaagtccgtgtgcatgaccttgttcctgctggctctgagggcgaggaatgagcacaggcaagctgatgagctggaggccatcatgcagggaaagggctctggcctgcagccagctgtttgcttggccatccgtgtcaacaccttcctcagctgcagtcagtaccacaagatgtacaggactgtgaaagccatcacagggagacagatttttcagcctttgcatgcccttcggaatgctgagaaggtacttctgccaggctaccaccactttgagtggcagccacctctgaagaatgtgtcttccagcactgatgttggcattattgatgggctgtctggactatcatcctctgtggatgattacccagtggacaccattgcaaagaggttccgctatgattcagctttggtgtctgctttgatggacatggaagaagacatcttggaaggcatgagatcccaagaccttgatgattacctgaatggccccttcactgtggtggtgaaggagtcttgtgatggaatgggagacgtgagtgagaagcatgggagtgggcctgtagttccagaaaaggcagtccgtttttcattcacaatcatgaaaattactattgcccacagctctcagaatgtgaaagtatttgaagaagccaaacctaactctgaactgtgttgcaagccattgtgccttatgctggcagatgagtctgaccacgagacgctgactgccatcctgagtcctctcattgctgagagggaggccatgaagagcagtgaattaatgcttgagctgggaggcattctccggactttcaagttcatcttcaggggcaccggctatgatgaaaaacttgtgcgggaagtggaaggcctcgaggcttctggctcagtctacatttgtactctttgtgatgccacccgtctggaagcctctcaaaatcttgtcttccactctataaccagaagccatgctgagaacctggaacgttatgaggtctggcgttccaacccttaccatgagtctgtggaagaactgcgggatcgggtgaaaggggtctcagctaaacctttcattgagacagtcccttccatagatgcactccactgtgacattggcaatgcagctgagttctacaagatcttccagctagagataggggaagtgtataagaatcccaatgcttccaaagaggaaaggaaaaggtggcaggccacactggacaagcatctccggaagaagatgaacctcaaaccaatcatgaggatgaatggcaactttgccaggaagctcatgaccaaagagactgtggatgcagtttgtgagttaattccttccgaggagaggcacgaggctctgagggagctgatggatctttacctgaagatgaaaccagtatggcgatcatcatgccctgctaaagagtgcccagaatccctctgccagtacagtttcaattcacagcgttttgctgagctcctttctacgaagttcaagtataggtatgagggaaaaatcaccaattattttcacaaaaccctggcccatgttcctgaaattattgagagggatggctccattggggcatgggcaagtgagggaaatgagtctggtaacaaactgtttaggcgcttccggaaaatgaatgccaggcagtccaaatgctatgagatggaagatgtcctgaaacaccactggttgtacacctccaaatacctccagaagtttatgaatgctcataatgcattaaaaacctctgggtttaccatgaaccctcaggcaagcttaggggacccattaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcggcttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagtttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatccttccagcatgtctctgcactaataaacagctaaattcacataatcatcctatttactgaagcatggtcatgctggtttatagattttttacccatttctactctttttctctattggtggcactgtaaatactttccagtattaaattatccttttctaacactgtaggaactattttgaatgcatgtgactaagagcatgatttatagcacaacctttccaataatcccttaatcagatcacattttgataaaccctgggaacatctggctgcaggaatttcaatatgtagaaacgctgcctatggttttttgcccttactgttgagactgcaatatcctagaccctagttttatactagagttttatttttagcaatgcctattgcaagtgcaattatatactccagggaaattcaccacactgaatcgagcatttgtgtgtgtatgtgtgaagtatatctgggacttcagaagtgcaatgtatttttctcctgtgaaacctgaatctacaagttttctgccaagccactcaggtgcattgcagggaccagtgataatggctgatgaaaattgatgattggtcagtgaggtcaaaaggagccttgggattaataaacatgcactgagaagcaagaggaggagaaaaagatgtctttttcttccaggtgaactggaatttagttttgcctcagatttttttcccacaagatacagaagaagataaagatttttttggttgagagtgtgggtcttgcattacatcaaacagagttcaaattccacacagataagaggcaggatatataagcgccagtggtagttgggaggaataaaccattatttggatgcaggtggtttttgattgcaaatatgtgtgtgtcttcagtgattgtatgacagatgatgtattcttttgatgttaaaagattttaagtaagagtagatacattgtacccattttacattttcttattttaactacagtaatctacataaatatacctcagaaatcatttttggtgattattttttgttttgtagaattgcacttcagtttattttcttacaaataaccttacattttgtttaatggcttccaagagcctttttttttttgtatttcagagaaaattcaggtaccaggatgcaatggatttatttgattcaggggacctgtatttccatgtcaaatgttttcaaataaaatgaaatatgagtttcaatactttttatattttaatatttccttaatattatggttattgtccgccattttgttgtatattgtaaataaagtttagattgt

SEQ ID NO: 2 - protein, 1043 aa, Homo sapiens
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 3 - DNA, 2414 bp, Homo sapiens
  CDS (163)..(1746)
actctctttacagtcagccttctgcttgccacagtcatagtgggcagtcagtgaatcttccccaagtgctgacaattaatacctggtttagcggcaaagattcagagaggcgtgagcagcccctctggccttcagacaaaaatctacgtaccatcagaaactatgtctctgcagatggtaacagtcagtaataacatagccttaattcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttcctgaagccagatatggtcattccattaatgtggtgtacagccgagggaaaagtatgggtgctctctttggaggacgctcatacatgccttctacccacagaaccacagaaaaatggaatagtgtagctgactgcctgccctgtgttttcctggtggattttgaatttgggtgtgctacatcatacattcttccagaacttcaggatgggctatcttttcatgtctctattgccaaaaatgacaccatctatattttaggaggacattcacttgccaataatatccggcctgccaacctgtacagaataagggttgatcttcccctgggtagcccagctgtgaattgcacagtcttgccaggaggaatctctgtctccagtgcaatcctgactcaaactaacaatgatgaatttgttattgttggtggctatcagcttgaaaatcaaaaaagaatgatctgcaacatcatctctttagaggacaacaagatagaaattcgtgagatggagaccccagattggaccccagacattaagcacagcaagatatggtttggaagcaacacgggaaatggaactgtttttcttggcataccaggagacaataaacaagttgtttcagaaggattctatttctatatgttgaaatgtgctgaagatgatactaatgaagagcagacaacattcacaaacagtcaaacatcaacagaagatccaggggattccactccctttgaagactctgaagaattttgtttcagtgcagaagcaaatagttttgatggtgatgatgaatttgacacctataatgaagatgatgaagaagatgagtctgagacaggctactggattacatgctgccctacttgtgatgtggatatcaacacttgggtaccattctattcaactgagctcaacaaacccgccatgatctactgctctcatggggatgggcactgggtccatgctcagtgcatggatctggcagaacgcacactcatccatctgtcagcaggaagcaacaagtattactgcaatgagcatgtggagatagcaagagctctacacactccccaaagagtcctacccttaaaaaagcctccaatgaaatccctccgtaaaaaaggttctggaaaaatcttgactcctgccaagaaatcctttcttagaaggttgtttgattagttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatatgagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaaaaaaaaaaaaaaaaaaaaaaaaaaaa

SEQ ID NO: 4 - protein, 527 aa, Homo sapiens
MSLQMVTVSNNIALIQPGFSLMNFDGQVFFFGQKGWPKRSCPTGVFHLDVKHNHVKLKPTIFSKDSCYLPPLRYPATCTFKGSLESEKHQYIIHGGKTPNNEVSDKIYVMSIVCKNNKKVTFRCTEKDLVGDVPEARYGHSINVVYSRGKSMGALFGGRSYMPSTHRTTEKWNSVADCLPCVFLVDFEFGCATSYILPELQDGLSFHVSIAKNDTIYILGGHSLANNIRPANLYRIRVDLPLGSPAVNCTVLPGGISVSSAILTQTNNDEFVIVGGYQLENQKRMICNIISLEDNKIEIREMETPDWTPDIKHSKIWFGSNTGNGTVFLGIPGDNKQVVSEGFYFYMLKCAEDDTNEEQTTFTNSQTSTEDPGDSTPFEDSEEFCFSAEANSFDGDDEFDTYNEDDEEDESETGYWITCCPTCDVDINTWVPFYSTELNKPAMIYCSHGDGHWVHAQCMDLAERTLIHLSAGSNKYYCNEHVEIARALHTPQRVLPLKKPPMKSLRKKGSGKILTPAKKSFLRRLFD

SEQ ID NO: 5 - DNA, 1816 bp, Homo sapiens
  CDS (40)..(825)
cttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaacccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacattaaaa

SEQ ID NO: 6 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 7 - protein, 24 aa, Artificial sequence
  Blocking peptide
SSKTTSVLQWAEKGYYTMSNNLVT

SEQ ID NO: 8 - protein, 13 aa, Artificial sequence
  Blocking peptide
QIAAHVISEASSK

SEQ ID NO: 9 - RNA, 22 nt, Artificial sequence
  RNA molecule
augucucugcagaugguaacnn

SEQ ID NO: 10 - RNA, 22 nt, Artificial sequence
  RNA molecule
cuguuaccaucugcagagacnn

SEQ ID NO: 11 - RNA, 22 nt, Artificial sequence
  RNA molecule
gguaggagaucuuccugaagnn

SEQ ID NO: 12 - RNA, 24 nt, Artificial sequence
  RNA molecule
ggggaugggcacuggguccaugnn

SEQ ID NO: 13 - RNA, 24 nt, Artificial sequence
  RNA molecule
agcauggacccagugcccauccnn

SEQ ID NO: 14 - RNA, 22 nt, Artificial sequence
  RNA molecule
cuguuaccaucugcagagacnn

SEQ ID NO: 15 - RNA, 24 nt, Artificial sequence
  RNA molecule
auggcagccucuuucccacccann

SEQ ID NO: 16 - RNA, 24 nt, Artificial sequence
  RNA molecule
ggugggugggaaagaggcugccnn

SEQ ID NO: 17 - RNA, 25 nt, Artificial sequence
  RNA molecule
aaacuugcagcucagcaaaaaacnn

SEQ ID NO: 18 - RNA, 26 nt, Artificial sequence
  RNA molecule
gaguuuuuugcugagcugcaaguunn

SEQ ID NO: 19 - RNA, 26 nt, Artificial sequence
  RNA molecule
gaguuuuuugcugagcugcaaguunn

SEQ ID NO: 20 - RNA, 24 nt, Artificial sequence
  RNA molecule
ucacaaaacccuggcccauguunn

SEQ ID NO: 21 - RNA, 24 nt, Artificial sequence
  RNA molecule
ggaacaugggccaggguuuugunn

SEQ ID NO: 22 - protein, 17 aa, Artificial sequence
  Pre-pro-insulin peptide
AGSLQPLALEGSLQKRG

SEQ ID NO: 23 - protein, 15 aa, Artificial sequence
  GAD peptide
PRLIAFTSEHSHFSL

SEQ ID NO: 24 - protein, 15 aa, Artificial sequence
  GAD peptide
FFRMVISNPAATHQD